(19) United States
(12) Patent Application Publication
(10) Pub. No.: US 2024/0008796 A1
(43) Pub. Date: Jan. 11, 2024

(54) METHOD AND SYSTEM FOR ANALYSING BRAIN ACTIVITY

(71) Applicant: INNONEURO LIMITED, KOWLOON (HK)

(72) Inventor: David Tibor Julian LILEY, Yarraville (AU)

(21) Appl. No.: 18/039,450

(22) PCT Filed: Oct. 12, 2021
(86) PCT No.: PCT/IB2021/059309

§ 371 Date: May 30, 2023

(30) Foreign Application Priority Data
Nov. 30, 2020 (HK) 32020021301.7

(51) Int. Cl. A61B 5/377; G16H 15/00; A61B 5/00

(52) U.S. Cl. CPC A61B 5/377; G16H 15/00; A61B 5/7235

(57) ABSTRACT

There is provided a method and system for constructing a representation of changes in the state of responsiveness of a mammalian subject's brain to a plurality of repeated external stimuli. The method and system include acquiring a plurality of brain activity measurements of a subject over a plurality of pre-determined time periods. Each of said measurements are of the subject's brain activity preceding and following the presentation of an external stimulus. The method and system further evaluate variability in the acquired brain activity measurements to a plurality of repeated external stimuli by a processor and generate a report of the changes in brain responsiveness states from the variability in said brain activity measurements over the plurality of predetermined time periods.

### FIELD

The present invention relates a method and system for analysing detected electrical signals from brain activity in response to repeated stimuli.

### BACKGROUND

In 1929 Hans Berger published a paper describing a device which could measure the electrical activity of the brain by placing electrodes on the scalp, amplifying the signal, and plotting the change in voltage over time in what is generally accepted as the first reported human electroencephalogram (EEG). Developments in numbers and location of electrodes, sampling, analytical and other techniques used in EEG have facilitated insights into brain activity, with discovery and understanding of various components of the signals providing a rich area of further research, including the identification of the first cognitive event related potential (ERP) component in the early 1960's. Significantly, surgical advances since Berger's original discovery have enabled the electroencephalogram to be recorded directly from the surface of the brain (electrocorticography or ECoG) or from electrodes implanted directly in the brain (stereo-electroencephalography or sEEG).

Other techniques have also been developed for observing brain activity over time in many areas, including investigating cognition, with such techniques including blood oxygen dependent (BOLD) functional magnetic resonance imaging (fMRI) and functional near infrared spectroscopy (fNIRS), as well as magnetoencephalography (MEG).

Notwithstanding the actual technique used, many investigative approaches for analysing brain activity employ a similar experimental protocol involving monitoring brain activity before, during and after presentation of one or more stimuli (visual, auditory or tactile/somatosensory) and quantifying either spontaneous or stimulus locked changes over a series of successive trials. One common experimental paradigm used is the “oddball paradigm”, where two classes of stimulus events are presented (rare and frequent/typical), with the class of stimulus determining the behavioural task the subject is required to perform. In analysing results of experiments using the “oddball paradigm”, and indeed also other experimental paradigms, detected electrical activity before, during and after each stimulus is tracked; filtered, and typically averaged across the numbers of trials; and the resultant readings compared and further evaluated.

When the brain activity associated with cognitive processes is investigated using these various techniques and approaches, the typical analytical paradigms reflect a set of assumptions (overt or otherwise) that require that distinct cognitive percepts corresponding to the stimuli, reflected in the measured change in electrical activity, are the result of invariant and distinct neurological processes. However, in each single session, even for the same individual, the event related responses recorded are highly variable, notwithstanding various types of pre-processing and artefact removal. Typically, such variability in the recorded activity is addressed by time ensemble averaging, with the variability dismissed as arising from “neurophysiological noise”. It would be appreciated that providing further insights into brain activity, especially as it pertains to cognitive processes, has diverse applications, including monitoring anaesthesia, detecting vigilance in individuals (e.g. heavy vehicle operators) and assessing individual longitudinal changes in brain function (disease).

Accordingly, it is an object of the present disclosure to address some of the problems and disadvantages of the previous approaches, and at least provide the public with further choice.

### SUMMARY

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. In accordance with a first aspect of the present invention, there is provided a method for constructing a representation of changes in the state of responsiveness of a mammalian subject's brain to a plurality of repeated external stimuli. The method may include:
(i) acquiring a plurality of brain activity measurements of a subject over a plurality of pre-determined time periods, wherein each of said measurements are of the subject's brain activity preceding and following the presentation of an external stimulus;
(ii) using a processor to evaluate variability in the acquired brain activity measurements to a plurality of repeated external stimuli; and
(iii) generating a report of the changes in brain responsiveness states from the variability in said brain activity measurements over the plurality of predetermined time periods.

The variability in said brain activity may be evaluated according to a probability density function

P_{X}_{ⱼ}_{(τ)}(xⱼ; τ)

for brain activity measurements corresponding to the random variable Xⱼ(τ), recorded at a plurality of physical brain locations, each indexed by the integer j, at a time τ, with respect to the presentation of stimuli at time τ=0.

Advantageously, the probability density function may be estimated from acquired brain activity measurements by using methods selected from the group comprising empirical cumulative distribution function method, scaled histogram estimate, and kernel density estimation. The brain activity measurements may be acquired by a modality selected from the group of modalities comprising electrocorticogram (ECoG), electroencephalogram (EEG), magnetoencephalogram (MEG), blood oxygen level dependent (BOLD) functional magnetic resonance (fMRI) and near infrared spectroscopy (NIRS).

Optionally, the stimuli are selected from the group comprising auditory, visual, olfactory or somatosensory.

The measurements may be acquired from a plurality of brain locations for a plurality of predetermined time periods. Advantageously, p_{X}_{ⱼ}_{(τ)}(xⱼ; T) is serially estimated in time from a plurality of sessions for a specified subject, wherein each session comprises measurements of brain activity for a plurality of repeated stimuli and the sessions are spaced apart at intervals selected from a plurality of hours, plurality of days, plurality of months or a plurality of years. The serial estimation of p_{X}_{ⱼ}_{(τ)}(xⱼ; τ) for a specified subject provides an indication of changes in brain function between sessions.

Optionally, the brain activity is time-ensemble estimated stimulus evoked activity, ERPⱼ(τ), which can be variously estimated as one of the following functions:
(i) ERPⱼ(τ)=E[Xⱼ(τ)], where E[·] is the expectation operator; or
(ii) ERPⱼ(τ)=median[Xⱼ(τ)]; or
(iii) ERPⱼ(τ)=mode[Xⱼ(τ)].

Optionally, p_{X}_{ⱼ}_{(τ)}(xⱼ; τ) is empirically estimated on frequency band limited brain activity measurements.

Advantageously, one brain responsiveness state is differential entropy determined according to the equation:

hⱼ(τ)=∫p_{X}_{ⱼ}_{(τ)}(xⱼ; τ)log p_{X}_{ⱼ}_{(τ)}(xⱼ; τ) dxⱼ

here hⱼ(τ) is the differential entropy at a time T after the presentation of a stimulus, for a physical brain location specified by the index j.

The method may further include: empirically estimating the differential entropy from a finite number of samples; and defining changes in differential entropy with respect to a baseline reference value.

The baseline reference value may include values of hⱼ(τ) for τ<0 (preceding stimulus presentation).

Optionally, the differential entropy may be estimated by using one of the techniques selected from the group comprising histogram-based bias correction estimation, kernel density estimation and k-nearest neighbour estimation.

Advantageously, hⱼ(τ) is longitudinally estimated from a plurality of sessions for a specified subject wherein each session comprises measurements of brain activity for a plurality of repeated stimuli and the sessions are spaced apart at intervals selected from a plurality of hours, plurality of days, plurality of months or a plurality of years.

Optionally, longitudinal estimates of hⱼ(τ) indexed by j are plotted topographically with respect to physical brain location.

The serial assessment of p_{X}_{ⱼ}_{(τ)}(xⱼ; τ) for a specified subject provides an indication of brain function over the cumulative sessions.

Advantageously, the variability of said brain activity is used to derive quantitative information theoretic measures representative of brain responsiveness state.

Optionally, the quantitative information theoretic measures representative of brain function are selected from the group comprising negentropy, differential entropy, “space averaged” differential entropy, Kullback-Leibler divergence and negentropy transfer entropy, mutual information, relative entropy and multiscale entropy.

The changes in brain responsiveness states are independent of corresponding time ensemble derived changes in ERP amplitude.

Optionally, the quantitative information theoretic measures representative of brain function may be longitudinally estimated from a plurality of sessions for a specified subject, wherein each session may comprise measurements of brain activity for a plurality of repeated stimuli and the sessions are spaced apart at intervals selected from a plurality of hours, a plurality of days, a plurality of months or a plurality of years.

Advantageously, the quantitative information theoretic measures representative of brain function longitudinally estimated from a plurality of sessions for a specified subject may provide an indication of brain function of the specified subject over the cumulative intervals.

Longitudinal estimates of quantitative information theoretic measures may be plotted topographically with respect to physical brain location, indexed by j.

In accordance with a second aspect of the present invention, there is provided a system for representing the changes in responsiveness states of a mammalian subject's brain in response to a plurality of repeated external stimuli, comprising:
(i) an acquiring module including a processor configured for acquiring a plurality of brain activity measurements of a subject over a plurality of pre-determined time periods, wherein each of said measurements are of the subject's brain activity preceding and following the presentation of an external stimulus;
(ii) an evaluating module including a processor configured for receiving said brain activity measurements and evaluating variability thereof over the plurality of the predetermined time periods; and
(iii) a determining module including a processor configured for determining changes in brain responsiveness states from the variability over the plurality of predetermined time periods and generating a report therefrom.

Optionally, the variability may be evaluated by a processor in the evaluating module configured to utilise a probability density function

P_{X}_{ⱼ}_{(τ)}(xⱼ; τ)

for brain activity corresponding to the random variable Xⱼ(τ), recorded at a plurality of physical brain locations, each indexed by the integer j, at a time τ, with respect to the presentation of stimuli at time τ=0.

The probability density function may be estimated from acquired brain activity measurements by using methods selected from the group comprising empirical cumulative distribution function method, scaled histogram estimate, and kernel density estimation.

Optionally, p_{X}_{ⱼ}_{(τ)}(xⱼ; τ) may be serially estimated in time from a plurality of sessions for a specified subject, wherein each session comprises measurements of brain activity for a plurality of repeated stimuli and the sessions are spaced apart at intervals selected from a plurality of hours, plurality of days, plurality of months or a plurality of years.

The serial estimation of p_{X}_{ⱼ}_{(τ)}(xⱼ; τ) for a specified subject may provide an indication of changes in brain function between sessions.

Optionally, one brain responsiveness state is differential entropy calculated by the processor of the determining module according to the equation:

hⱼ(τ)=∫p_{X}_{ⱼ}_{(τ)}(xⱼ; τ)log p_{X}_{ⱼ}_{(τ)}(xⱼ; τ) dxⱼ

where hⱼ(τ) is the differential entropy at a time T after the presentation of a stimulus, for a physical brain location specified by the index j.

In accordance with a third aspect of the present invention, there is provided a computer readable medium comprising program instructions that, when executed by one or more processors to implement the method discussed above.

### BRIEF DESCRIPTION OF FIGURES

In order to describe the manner in which the above-recited and other advantages and features of the disclosure can be obtained, a more particular description of the principles briefly described above will be rendered by reference to specific embodiments thereof which are illustrated in the appended figures. Understanding that these figures depict only exemplary embodiments of the disclosure and are not therefore to be considered to be limiting of its scope, the principles herein are described and explained with additional specificity and detail through reference to the accompanying figures.

In particular, preferred embodiments of the present disclosure will be explained in further detail below by way of examples and with reference to the accompanying figures, in which:

FIG. 1A depicts an exemplary schematic representation of the typical prior art approach of interpreting EEG waveforms using time ensemble averaging, typically for a single electrode, to remove variability in a series of epochs for a single individual.

FIG. 1B depicts an exemplary schematic representation of the typical prior art approach interpreting EEG waveforms using time-frequency analysis for a series of epochs for a single electrode and individual.

FIG. 2A depicts an exemplary arrangement of hardware in a system for recording event related responses to visual stimuli.

FIG. 2B depicts an exemplary schematic arrangement of an embodiment of the processing system of the present disclosure.

FIG. 3 depicts a schematic representation of an approach of interpreting EEG waveforms using a probability distribution function to analyse the variability for a series of epochs for a single individual for a single electrode/sensor/channel/brain location.

FIG. 4A depicts a topographic map of the change in differential entropy, with respect to a pre-stimulus baseline, for a representative subject using baseline uncorrected epochs, at a latency of 0.39 s post-stimulus for multichannel EEG referenced to a common average electrode average (Common Average data); together with subplots of differential entropy, hⱼ(τ), and the Kullback-Leibler divergence, D_{KY}[Xⱼ(τ>0)∥Xⱼ(τ≤0)], for channels (labelled with respect to the extended 10-290 system of electrode placement) (i) PO5, (ii) PO6 and (iii) POz in accordance with Example 1.

FIG. 4B depicts a topographic map of the change in the ensemble averaged ERP amplitude for a representative subject using baseline uncorrected epochs, at a latency of 0.39 s post-stimulus for Common Average data; together with subplots of ERP amplitude and negentropy for channels (i) PO5, (ii) PO6 and (iii) POz in accordance with Example 1.

FIG. 4C depicts a topographic map of the change in differential entropy for a representative subject using baseline uncorrected epochs, at a latency of 0.39 s post-stimulus for multichannel scalp current density (SCD) EEG data (Laplacian-referenced); together with subplots of differential entropy, hⱼ(τ), and the Kullback-Leibler divergence, D_{KY}[Xⱼ(τ>0)μXⱼ(τ≤9)], for channels (i) PO5, (ii) PO6 and (iii) POz in accordance with Example 1.

FIG. 4D depicts a topographic map of the post-stimulus change in ensemble averaged ERP amplitude for a representative subject using baseline uncorrected epochs, at a latency of 0.39 s post-stimulus for Laplacian-referenced data; together with subplots of negentropy for channels (i) PO5, (ii) PO6 and (iii) POz in accordance with Example 1.

FIG. 5A depicts a topographic map of the differential entropy for the representative subject of FIG. 4A, this time using zero-meaned data, at a latency of 0.39 x post-stimulus for Common Average data; together with subplots of differential entropy, hⱼ(τ), and the Kullback-Leibler divergence, D_{KY}[Xⱼ(τ>0)∥Xⱼ(τ≤0)], for channels (i) PO5, (ii) PO6 and (iii) POz.

FIG. 5B depicts a topographic map of the change in the ensemble averaged ERP amplitude for the representative subject of FIG. 4B, this time using zero-meaned data, at a latency of 0.39 s post-stimulus for Common Average data; together with subplots of ERP amplitude and negentropy for channels (i) PO5, (ii) PO6 and (iii) POz.

FIG. 5C depicts a topographic map of the change in differential entropy for the representative subject of FIG. 4C, this time using zero-meaned data, at a latency of 0.39 s post-stimulus for Laplacian-referenced data; together with subplots of differential entropy, hⱼ(τ), and the Kullback-Leibler divergence, D_{KY}[Xⱼ(τ)∥Xⱼ(τ₀)], for channels (i) PO5, (ii) PO6 and (iii) POz.

FIG. 5D depicts a topographic map of the post-stimulus change in ensemble averaged ERP amplitude for the representative subject of FIG. 4D, this time using zero-meaned data, at a latency of 0.39 s post-stimulus for Laplacian-referenced data; together with subplots of ERP amplitude and negentropy for channel s (i) PO5, (ii) PO6 and (iii) POz.

FIG. 6A depicts bias corrected differential entropy, hⱼ(τ), for three selected channels for the same subject of FIG. 4A to FIG. 4D and FIG. 5A to FIG. 5B, that includes the channel (O1) that has the maximal change in entropy following the presentation of the stimulus.

FIG. 6B depicts the distribution of the minimum differential entropy across all channels.

FIG. 7A to FIG. 7H depict plots of bias corrected spatially-averaged differential entropy, H(t), and directional variance, (dva), for eight subjects for baseline uncorrected Common Average data.

FIG. 8A to FIG. 8H depict plots of bias corrected spatially averaged differential entropy, H(t), and directional variance, (dva), for eight subjects for zero-meaned Common Average data.

FIG. 9A depicts the bias corrected spatially averaged (across all electrodes/sensors/channels/brain locations) differential entropy, together with bootstrap calculated confidence intervals, across all participants in the experiment of Example 1.

FIG. 9B depicts the directional variance (dva) together with bootstrap calculated confidence intervals, across all participants in the experiment of Example 1.

FIG. 10A depicts the correlations between ensemble averaged ERP amplitude and differential entropy, across all stimulus latencies and electrodes/sensors/channels/brain locations, for the same subject of FIG. 4A to FIG. 4D and FIG. 5A to FIG. 5D.

FIG. 10B depicts the spatially averaged differential entropy and directional variance (dva) for the same subject of FIG. 4A to FIG. 4D and FIG. 5A to FIG. 5D.

FIG. 11 depicts an exemplary division of electrodes into sagittal and lateral groups as utilised in Example 2.

FIG. 12 depicts exemplary lexical neighbourhood ERP amplitude plots, according to the division of electrode groups in FIG. 11, arising from the experiment described in Example 2.

FIG. 13 depicts exemplary semantic features ERP plots arising from the experiment described in Example 2.

FIG. 14 depicts exemplary lexical neighbourhood differential entropy plots arising for the experiment described in Example 2.

FIG. 15 depicts semantic features differential entropy plots arising for the experiment described in Example 2.

FIG. 16 depicts lexical neighbourhood negentropy plots arising for the experiment described in Example 2.

FIG. 17 depicts semantic features negentropy plots arising for the experiment described in Example 2.

### DETAILED DESCRIPTION OF THE PREFERRED EMBODIMENTS

Various embodiments of the disclosure are discussed in detail below. While specific implementations are discussed, it should be understood that this is done for illustrative purposes only. A person skilled in the relevant art will recognize that other components and configurations may be used without departing from scope of the disclosure.

In investigations of brain activity, one or more stimuli (visual, auditory or tactile/somatosensory) are presented and the electroencephalograph (EEG) response of the brain to these stimuli is recorded.

Such electrical activity is referred to as Event Related Brain Activity, and by assuming it is uncorrelated with the background ongoing neural activity and noise artefacts (caused by activity such as eyeblinks, eye movement and electrical mains interference) can be resolved/extracted by using some type of signal processing, typically averaging across the responses to multiple stimulus presentations (trials), as detailed further below.

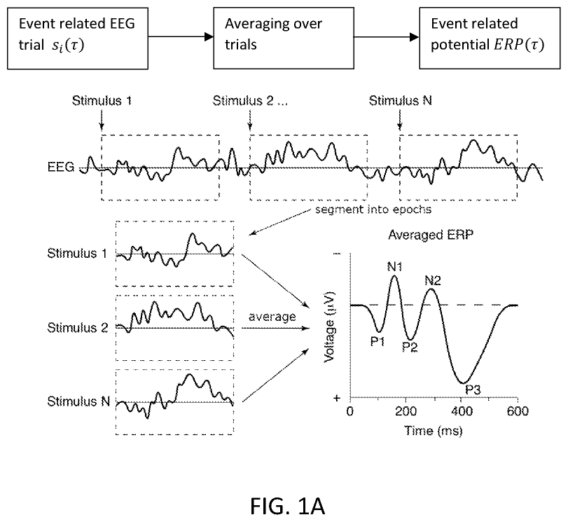

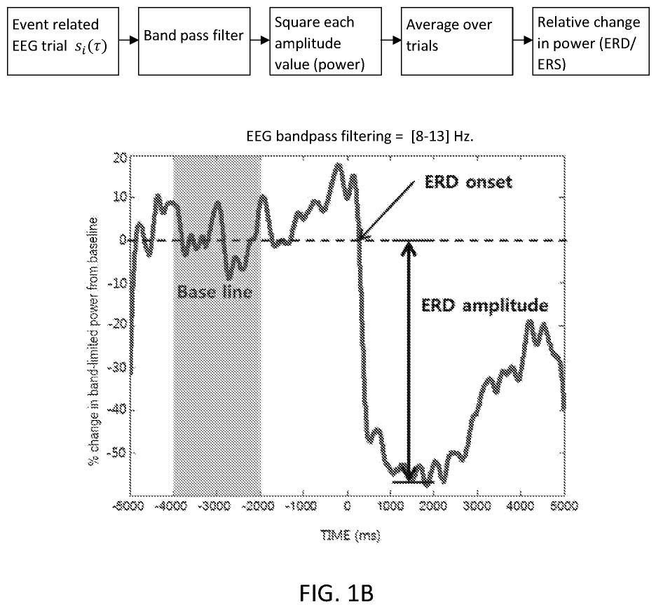

The standard experimental paradigm involves recording a subject's response to multiple presentations of the same stimulus over a number of different time intervals or epochs, the final response presented usually being an average representation of the individual responses over a predetermined time interval, aligned to the time of stimulus presentation. A schematic representation of this standard experimental paradigm is depicted in FIG. 1A.

Such recorded, stimulus induced, electrical activity is classified into two main types, being evoked activity and induced activity.

Evoked activity is a time domain activity which is time and phase locked to stimulus onset; and is calculated by the averaging of the responses of individual trials to the repeated presentation of a stimulus with the signal-to-noise ratio of the evoked response increasing as the square root of the number of trial averages. Thus, as the number of trials is doubled, the noise is decreased by about 30%; and to reduce the noise by half it is necessary to have four times as many trials.

The resulting voltage fluctuations are generally referred to as Event Related Potentials (ERPs). The principle theoretical assumptions underpinning this process have been comprehensively described by Rugg and Coles (Event-related brain potentials: An introduction. In M. D. Rugg & M. G. H. Coles (Eds.), Oxford psychology series, No. 25. Electrophysiology of mind: Event-related brain potentials and cognition (p. 1-26). Oxford University Press, 1995) as:
1. the signal, underlying the response of interest, is invariable in latency and shape, consisting of an event locked epoch of ERPs as depicted in FIG. 1A.
2. the signal of interest is independent from the ongoing background activity, and is thus not expected to vary from trial to trial.

By contrast, induced activity is time locked but not phase locked to the stimulus onset. In other words, induced changes in EEG amplitude have a variable trial-to-trial time course of onset from trial-to-trial following stimulus presentation. In comparison evoked changes in EEG amplitude will have a consistent trial-to-trial time course of onset following stimulus presentation. Induced activity is calculated in terms of the trial-averaged percentage change in short-time windowed spectral power, for one or more frequency bands, as a function of time following stimulus presentation.

These changes in oscillatory power over time, depending on the relative magnitude of their change are typically referred to as Event Related Desynchronization (if band oscillatory power increased relative to a pre-stimulus baseline) (ERD) or Event Related Synchronization (if band oscillatory power decreased relative to a pre-stimulus baseline) (ERS). Such changes are often grouped together and referred to as an Event Related Spectral Perturbation (ERSP; e.g. Makeig et al. Mining event-related brain dynamics. Trends in Cognitive Science 1995;8:204-210).

Both induced activity and evoked activity have been utilised to better understand how the brain processes stimuli as well as being used as the basis for attempts to monitor brain function in health (e.g. awareness/vigilance), during medical intervention (e.g. anaesthetic effect) and in disease (e.g. dementia, schizophrenia, depression).

Both of the above standard processing approaches can be used in the longitudinal assessment of brain function, but do not detect an individual's unique pattern, or signature, of brain activity.

Therefore, according to the state of the art, there are two options available to quantify EEG activity in response to a stimulus:

### Option 1: Time Ensemble Averaging to Obtain Evoked Activity

Perform some form of time-ensemble averaging of the extracted data/measurement segments aligned to stimulus onset (i.e. τ=0) to obtain ERPs, or some other event related response depending on the modality chosen, on the basis of an additive noise mode according to the following function:

$$\begin{matrix}{s}_{i} ( τ ) = {r}_{i} ( τ ) + {e}_{i} ( τ ) \\ = r ( τ ) + e\end{matrix} \qquad ( 1 )$$

where i indexes the i-th stimulus presentation, sᵢ(τ) is the recorded response for the i-th stimulus presentation as a function of stimulus latency τ, r(τ) is the assumed underlying, phase locked, trial invariant stimulus response at latency τ (i.e. the ERP) and e is a zero mean independent and identically distributed (i.i.d.) additive noise of variance σₑ² (that by definition is independent of trial and stimulus latency) assumed to arise from uncorrelated background, electroencephalographic or otherwise, activity.

On this basis the expected value of sᵢ(τ), E[sᵢ(τ)], is r(τ) (i.e. the ERP) while the variance of the trial average is Var[1/N Σᵢ₌₁^{i=N} sᵢ(τ)]=Var[e]/N≡σₑ²/N, such that the signal-to-noise ratio (SNR) of the time ensemble extracted ERP is \sqrt{N}r(τ)/σₑ i.e. the SNR of the extracted ERP increases as the square root of the number of trials averaged.

Optionally, in a typical EEG stimulus processing paradigm, the analysis described above may be performed in addition or alternative to Option 2 described below.

### Option 2: Time Frequency Analysis to Obtain Induced Activity

Perform, using one of many available methods (e.g. windowed short time FFT, continuous or discrete wavelet transformation), time-frequency analysis on each extracted segment before ensemble averaging to obtain ERS/ERD/ERSP activity (typically in terms of the percentage change from a pre-stimulus baseline, though other baselines can be used for example z-normalizing (standard scoring) event related spectra to a pre-stimulus baseline) for a given frequency band. Typical EEG frequency bands over which such induced activity can be calculated include the classically defined human EEG bands of delta (typically 0-4 Hz), theta (typically 4-8 Hz), alpha (typically 8-13 Hz), beta (typically 13-30 Hz) and gamma (typically >30 Hz) or any other, typically narrow, frequency band.

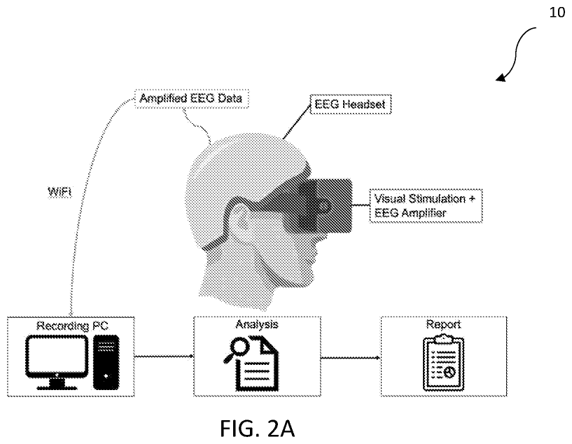

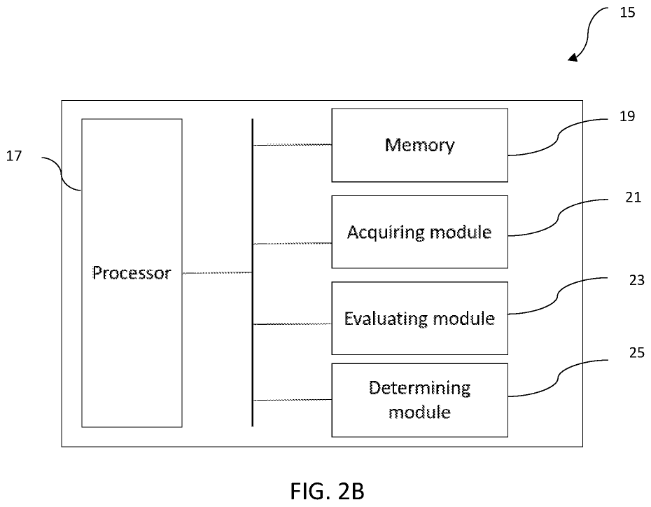

It should be appreciated that by design, the standard approach to ERP is to destroy variability information (typically dismissed as “neurophysiological noise”) by obtaining the relevant average across successive trials. Exemplary stages of this approach are schematically depicted in FIG. 1B, as well as an exemplary ERD (in this case an alpha band ERSP) derived from this approach. FIG. 2A describe the typical components in a system 10 which may employ the method and approach of the present disclosure using an EEG measurement modality for reference. It would be appreciated by persons skilled in the art that a similar arrangement (modified with respect to each sensing modality as required) could be used for other sensing modalities under investigation. Many of the steps of our method will be familiar to persons skilled in the art of recording brain activity, and are not discussed further herein.

### 1. Choice of Stimuli

Potentially any visual, auditory, tactile/somatosensory or gustatory/olfactory stimulus or class of stimuli (e.g. pictures of concrete nouns, spoken words, visual checkerboard stimuli, brief exposure to odourants etc.) can be chosen and presented to the subject without departing from the scope of the present disclosure.

From a practical perspective, stimuli or sets of stimuli are chosen, either on the basis of previous studies involving ERP or ERD/ERS/ERSP analysis, or on the basis of neuropsychological requirements, that are expected to probe specific aspects of cognitive function (e.g. memory, attention, semantic cognitive processing or similar).

One example could be a visual memory task, involving the presentation of a sequence of visual images, at semi-regular short intervals, in which there are one or more repeated stimuli, in order to investigate or to probe short term memory.

As set out in Example 1 below, an exemplary experiment could require subjects to participate in a silent reading task, in which single words are briefly presented, each followed by a black (“blank”) screen; with subjects being asked to read the words silently to themselves. Responses could then be recorded under a high or low memory load (by requiring subjects to memorize a new sequence of six random integers (high load) or six identical integers (low load) every 8-12 word presentations) to investigate the electrophysiological correlates of the cognitive processing of words in responses to a systematic variation in background cognitive activity.

Another exemplary experimental arrangement is described in Example 2 below. It would be appreciated that the specifics of the experimental arrangement are not critical to the present disclosure, which is directed to the interpretation of the results obtained.

Typically, as is the case in collecting ERP (“evoked”) and ERD/ERS/ERSP (“induced”) electroencephalographic responses, stimuli are presented sequentially separated by time intervals judged to be sufficiently long to capture the time course of the single trial “evoked”/“induced” response. Similar considerations would apply to the other monitoring modalities considered.

Stimulus duration is typically kept as short as possible subject to 1) ensuring that an appropriate response will be elicited (with respect to Example 1, this will be electroencephalographic) and 2) whether “conscious” stimulus processing is to be mitigated.

For visual stimuli it is typical to display such stimuli for between ˜50 ms-500 ms, and to present stimuli every ˜1.5 s-2.5 s. However, there are many variations to presentation sequencing, stimulus contingencies and timings familiar to those skilled in the art of recording brain activity in response to stimulus presentation for electroencephalographic monitoring. After stimulus presentation, depending on the requirements of the task, participants may be required to make some form of vocal or motor response e.g. depressing a specific button etc.

In many cases it will be necessary to normalize stimulus presentation to one or more perceptual thresholds in order to account for the inevitable variations in visual, auditory or somatosensory functioning in health (e.g. presbycusis/presbyopia) or disease.

2. Recording of response

Before and during stimulus presentation the brain's electrical activity, electrical (EEG, MEG, ECoG) or otherwise (BOLD fMRI, NIRS), may be recorded in order to measure the response of the brain to the stimulus. Because the brain will exhibit considerable topographic/spatial variability in its response such recording will need to occur at multiple, and widely distributed, brain locations.

In the case of recording the brain's electrical activity, this is typically achieved by, but not restricted to, recording the EEG according to a standardized system for scalp electrode (wet or dry, active or passive) placement (e.g. the 10-20 system which accommodates 21 scalp locations, the extended 10-20 (also referred to as the “10%” or 10-10 systems) which can accommodate up to 74 electrodes or the 10-5 or “5%” system which specifies up to 345 electrode locations), appropriately referenced and grounded.

For the EEG the fidelity and quality of the recorded signal are typically determined by digitization depth (12-24 bits), sampling frequency (˜80-5000 Hz) and the ambient electrical environment (which the current state of the art mitigates through the utilization of active electrode configurations).

For later analysis the time of stimulus onset must be recorded contemporaneously with the recorded signal through an appropriate single/multi-bit/word trigger.

This is typically achieved by the stimulus presentation computer sending (through an appropriate configured serial or parallel port) a trigger, at stimulus onset, to the computer running the brain activity measurement acquisition system.

### 3. Pre-Processing of Recorded Response

Following the single/multi-channel recording of the response to a large number (in the case of EEG & MEG typically >˜30-40) of sequentially presented stimuli (the response to each presentation being typically referred to as a trial) the continuous time series for each spatial location (e.g. each EEG recording electrode) needs to be segmented about the times of the onset of each stimulus.

This process is typically referred to as “epoching” the data.

Before “epoching occurs, various filtering techniques may be applied to the data/measurements to remove adventitious noise (in the case of EEG most notably 50/60 Hz mains artefact; or in the case of BOLD fMRI ballistocardiogram and subject movement) as well as endogenous physiological artefact (most significantly eyeblink induced electrical activity).

Following such filtering, “epoching” involves extracting data segments aligned to the time of stimulus onset. These data/measurement segments extend from sometime prior (e.g. for EEG/MEG˜100ms-500 ms) to stimulus onset to sometime post (e.g. for EEG/MEG ˜1000 ms-2000 ms) stimulus onset and are temporally aligned such that the time of stimulus onset (τ) is defined as τ=0 i.e. for EEG/MEG the data/measurement segment (or epoch) would typically be defined as extending over the interval τ=[−500 to −100, ˜1000 to 2000] ms. It would be appreciated that other predetermined epochs, or time intervals could be utilized.

Times after stimulus presentation are defined as positive latencies (with respect to stimulus presentation) i.e. ϵ>0, whereas times before stimulus presentation are defined as negative latencies (with respect to stimulus presentation) i.e. τ<0.

Following such data/measurement segmentation individual epochs may be inspected, or otherwise processed, to ensure the absence of any endogenous or exogenous artefact.

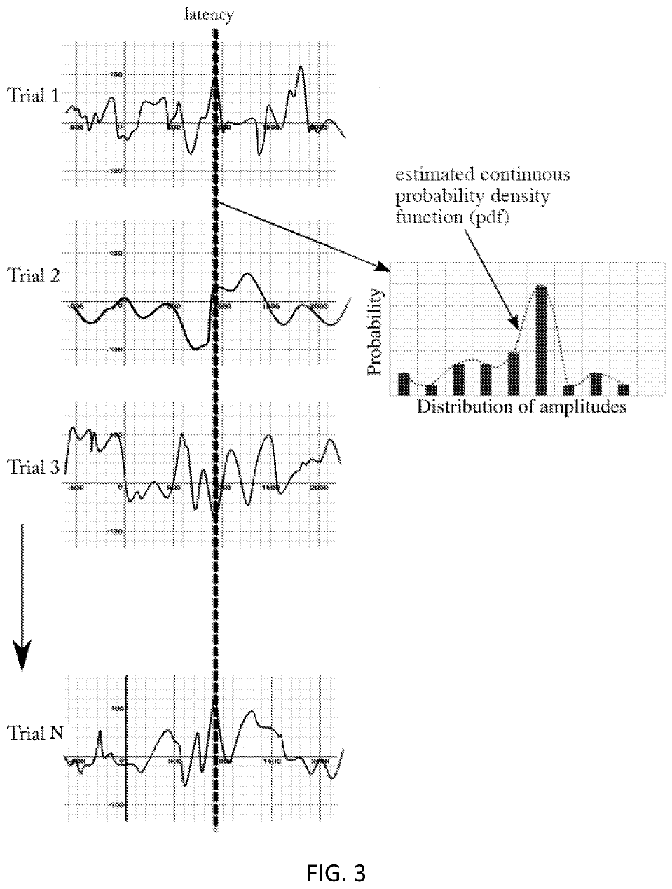

At this point the state of the art would be to calculate some form of averaged response, as described above with reference to FIG. 1A (adapted from Luck et al. Trends in Cognitive Sciences 2000;4(1):432) and FIG. 1B (adapted from Park et al. Journal of Motor Behaviour 2018;50(4):457), representative of the stimulus produced brain activity, thereby eliminating all trial-to-trial variability. However, in our present disclosure and as is discussed below with reference to FIG. 3, an important departure is made from the approaches used to calculate evoked and induced activity.

A schematic representation of an exemplary embodiment of the analytical system 15 is depicted with reference to FIG. 2B.

One or more processors 17 are configured to perform the steps described below to analyze the measurements of brain activity according to instructions stored in memory 19.

Specifically, the acquiring module 21 is configured to acquire measurements of brain activity of a subject over a plurality of pre-determined time periods and electrodes/sensors/channels/brain locations, wherein the predetermined time periods have a duration selected to include brain activity preceding and following the presentation of the plurality of repeated external stimuli to the subject.

An evaluating module 23 is configured for evaluating variability in said brain activity measurements obtained over a plurality of the predetermined time periods.

A determining module 25 is configured for determining changes in brain responsiveness states from the variability over the plurality of predetermined time periods.

As depicted in FIG. 3, a subject's response to multiple presentations of the same stimulus over a number of different time intervals or epochs is recorded. It is at this point, also with reference to FIG. 3, that an important departure is made from the traditional approaches used to calculate evoked and induced activity, where trial-to-trial variability is retained in order to enable the development of an important method for characterizing the brain function of both a single individual and a corresponding population of subjects.

Advantageously, this may be done by empirically constructing/estimating, a probability density function (probability distribution) for the j-th electrode/sensor's EEG amplitude (or at a particular brain location for any other brain-related signal) Xⱼ(τ), p_{X}_{ⱼ}_{(τ)}(xⱼ; τ), as a function of stimulus latency τ. Here Xⱼ(τ) is the random variable corresponding to the EEG amplitude (or any other brain related signal as previously discussed) at a fixed latency T with respect to stimulus presentation (τ=0).

Empirical determinations of the probability density functions can be made using a number of methods that include binned histogram estimates and kernel density estimation methods.

On this basis the ERP, specified for EEG or MEG, at the j-th electrode/sensor can be specified as rⱼ(τ)=E[Xⱼ(τ)] or alternatively as rⱼ(τ)=median[Xⱼ(τ)] or rⱼ(τ)=mode [Xⱼ(τ)], depending on the properties of the distribution p_{X}_{ⱼ}_{(τ)}(xⱼ; τ), which may be known.

Significantly, in this approach there is no need to assume the existence of an independently and identically distributed additive noise process as there is no restriction that the variance of the recorded signal, Var[Xⱼ(τ)], be independent of stimulus presentation latency T.

The present disclosure has identified that the removal of this restriction enables an experimenter to define stimulus actuated electroencephalographic, responses (or any other measured brain activity according to the measurement modality utilized), in terms of a variety of information theoretic measures, including but not limited to: differential entropy, transfer entropy, relative entropy/Kullback-Leibler divergence, negentropy and multiscale entropy.

Accordingly, the present disclosure, hypothesizes that the variance, or any other measure of dispersion, must in fact change following stimulus presentation when calculated over repeated stimulus presentations; rather than remain invariant. The expectation that such variance doesn't change underlies the common approach of the prior art to time ensemble averaging of readings from successive trials.

Biologically speaking when an organism reacts to a given stimulus in the environment it should be associated with a reduction in the possible repertoire of the associated behavioral response. If there was no reduction in the possible repertoire of associated behavioral response, then it could be argued that the stimulus had no meaning to the organism.

When understood electrophysiologically, in the context of higher human cognition, the present disclosure identifies that the presentation of meaningful time locked stimuli should be associated with a reduction in the uncertainty of the subsequently evoked neurophysiological response. Thus, for stimuli known to be meaningful the absence of any reduction in subsequent variability of the electrophysiological response can only mean that either in this particular case the stimulus had no received meaning and/or the measured evoked activity is not causally (informationally) relevant to any subsequent cognitive response.

This may be contrasted with the standard additive noise model of the prior art, which is premised upon the assumption that stimulus evoked activity cannot be associated with any reduction in uncertainty, as trial variance with respect to latency remains constant after the presentation of the stimulus. That is, the actual evoked response is trial invariant. Behaviorally this makes no sense, and the present disclosure has identified that this premise is biologically untenable.

Referring to FIG. 3 and appreciating the inappropriateness of the underlying assumption of the prior art further aspects of the present disclosure can be appreciated.

Following the segmentation of a continuous EEG recording, for a given electrode/sensor/channel/brain location j, about the time of stimulus onset probability density function estimates of brain activity for a given latency τ, p_{X}_{ⱼ}_{(τ)}(xⱼ; τ), can be constructed using a variety of methods. For example, such methods include by way of exemplary non-limiting example, 1) empirical distribution function estimation 2) scaled histogram estimation and 3) kernel density estimation; methods all well described and familiar to persons skilled in the art with knowledge of empirical estimation of probability density functions.

For any system adapted in some way to its environment, like the human brain, the variance, but more specifically the differential entropy (a measure of the average surprisal of a continuous random variable), hⱼ(τ)

hⱼ(τ)=∫p_{X}_{ⱼ}_{(τ)}(xⱼ; τ) log p_{X}_{ⱼ}_{(τ)}(xⱼ; τ) dxⱼ (2)

must decrease transiently, from its pre-stimulus value, as determined on the basis of multiple stimulus presentations over some short period of time (a period of time sufficiently short such that the “meaning” of the stimulus is expected to remain invariant).

Indeed, as specified in Example 1, analysis of time-locked electroencephalographic activity in response to a passive reading task (wherein the time of stimuli presentation is defined as the onset of sequentially presented single words displayed for 500 ms, separated by a 1200 ms inter-stimulus interval) unequivocally indicates that numerical estimates of hⱼ(τ) transiently decrease following stimulus presentation which are topographically heterogeneous. Further, as specified in Example 2, such changes may be used to differentiate between the lexical (orthographic) and semantic features of such visually presented word stimuli.

### EXAMPLE 1

### Detecting Stimulus Activate Changes:

Example 1 demonstrates the use of the so defined probability density function p_{X}_{ⱼ}_{(τ)}(xⱼ; τ) in the calculation of the informational measure hⱼ(τ) (differential entropy) in an exemplary experimental process.

A. Participants and Task

| Participants | 16 (8 males and 8 females) | |
|---|---|---|
| Task | A sequence of words, each visible for 500 ms and separated by | |
| | 1200 ms, with participants instructed to passively read. | |
| Modifying | i) | High memory load condition: participants were required |
| conditions | | to memorise a sequence of 6 randomly generated |
| | | integers every 8-12 word presentations. |
| | ii) | Low memory load condition: participants were required |
| | | to memorise a new sequence of 6 identical integers |
| | | every 8-12 word presentations. |

The mean number of trials for both high and low memory conditions was 722.77 (SD 44.17) trials or approximately 361 per condition based on a 50:50 presentation contingency.

B. EEG Recording and Preprocessing

| | |
|---|---|
| Equipment | Neuroscan 64 channel SynAmps2/RT EEG recording system |
| Sensor location | Extended 10-20 electrode placement (64 channel Quik-Cap ™) |
| Recording | 1000 Hz sampling rate |
| configuration | common average referencing |
| | all electrode impedance <5 kΩ |
| | bandpass filtering 0-35 Hz |
| Data | artefact rejection (visual and independent components |
| preprocessing | analysis) performed using the FieldTrip software toolbox |
| | http://www.fieldtriptoolbox.org/ for MATLAB |
| | data/measurement epochs were extracted spanning the time |
| | period 0.3 s before stimulus onset to 1.4 s after stimulus |

Trials with noticeable artefact remaining after independent components analysis were rejected based on visual inspection. Analyses occurred with both zero-meaned and baseline uncorrected epochs on common average (CA) referenced data/measurements and estimated scalp current density (SCD). No other form of baseline correction was performed. Estimates of SCD were computed using the spherical spline method as implemented by ft_scalpcurrentdensity in FieldTrip.

C. Calculation of the Information Theoretic Measures Including Differential Entropy

For the j-th electrode/position an amplitude distribution is formed across trials for each fixed stimulus latency τ to empirically estimate the probability density p_{X}_{ⱼ}_{(τ)}(xⱼ; τ) in order to calculate the differential entropy. For this continuous probability density the differential entropy, hⱼ(τ), is defined as

hⱼ(τ)=∫p_{X}_{ⱼ}_{(τ)}(xⱼ; τ) log p_{X}_{ⱼ}_{(τ)}(xⱼ; τ) dxⱼ (3)

However, because the number of epochs is finite, p_{X}_{ⱼ}_{(τ)}(xⱼ; τ) will have to be estimated from binned amplitude data/measurements. Thus, assuming the random amplitude Xⱼ(τ) at time τ is partitioned into bins of width A we calculate the differential entropy hⱼ(τ) as

$$\begin{matrix}{h}_{j}^{Δ} ( τ ) = {H}_{j}^{Δ} ( τ ) + log Δ \\ = - {\sum }_{i} Δ {p}_{{X}_{j} ( τ )} ( i Δ ; τ ) log Δ {p}_{{X}_{j} ( τ )} ( i Δ ; τ ) + log Δ \\ = - {\sum }_{i} Δ {p}_{{X}_{j} ( τ )} ( i Δ ; τ ) log {p}_{{X}_{j} ( τ )} ( i Δ ; τ )\end{matrix} \qquad ( 4 )$$

where Hⱼ^{Δ}(τ) is the Shannon entropy of the discrete probability distribution p_{X}_{ⱼ}_{(τ)}(iΔ; τ). However, this simple plug-in estimate of entropy is well known to be a biased estimate in that it underestimates the true entropy. There are a number of ways to correct for this bias, among them that of bootstrap bias correction. Because this bootstrap correction also allows the calculation of 95% confidence intervals we choose this method for our bias correction. Our bootstrap bias corrected differential entropy, ĥⱼ^{Δ}(τ), is calculated as

$${\hat{h}}_{j}^{Δ} ( τ ) = 2 {h}_{j}^{Δ} ( τ ) - \frac{1}{B} {\sum }_{b = 1}^{B} {{h}_{j}^{Δ} ( τ )}^{b} \qquad ( 5 )$$

where B is the number of bootstrap samples and hⱼ^{Δ}(τ)^{b} is the differential entropy of the b-th bootstrap sample calculated using the plugin-estimator of the above equation (5) . By choosing B=1000 we obtain our 95% confidence intervals by rank ordering the hⱼ^{Δ}(τ)^{b} and choosing the lower 2.5% and upper 97.5% percentile boundaries. Topographic maps of ĥⱼ^{Δ}(τ) were plotted with respect to a pre-stimulus baseline. Specifically, baseline changes in differential entropy

δ\hat{h}ⱼ^{Δ}(τ)≡\hat{h}ⱼ^{Δ}(τ)−\hat{h}_{0,j}^{Δ}(τ<0) (6)

were plotted for −0.3<τ<1.4 s with ĥ_{0,j}(τ<0) the differential entropy calculated on the pre-stimulus amplitude distribution formed over the time interval of −0.3 to 0 s. Calculating such baseline changes also mitigates some undesirable properties of differential entropy (boundedness and negativity) compared to its discrete (Shannon Entropy) counterpart. In general, such changes will depend upon properties of the stimulus or classification of stimuli over which such a deviation is calculated i.e. δĥⱼ^{Δ}(τ|stimulus). Ensemble averaged ERPs with respect to a pre-stimulus baseline are also topographically plotted. In order to illustrate the particular features of the estimated differential entropy we compare it to a measure of variability, referred to as directional variance (dva) that has been used, notably by Schurger et al. (Cortical activity is more stable when sensory stimuli are consciously perceived. PNAS 2015;112(16):E2083-E2092), that is calculated using all recording electrodes/sensors/channels/brain locations. Directional (circular or angular) variance (dva), calculated only on scalp current density derived data/measurements such that orthogonality of channel data/measurements can be better justified, is defined as,

$$dva ( τ ) = 1 - R ( τ ) \\ R ( τ ) = \frac{1}{N} ‖ {\sum }_{i = N}^{i = N} \frac{{v}_{i} ( τ )}{‖ {v}_{i} ( τ ) ‖} ‖ \qquad ( 7 )$$

where vᵢ(τ)=[x₁(τ), . . . , x_{M}(τ)]ᵢ is the vector of M channels of time locked EEG amplitudes at stimulus latency T for the i-th epoch. R(τ) is often referred to as the directional coherence. Specifically, dva was compared with the estimated ĥⱼ^{Δ}(τ) averaged over all electrodes.

In addition, the information theoretic quantities for individual channels, the Kullback-Leibler divergence (relative entropy) D_{KL}[Xⱼ(τ)>0∥Xⱼ(τ≤0)] and negentropy were also calculated. The negentropy, a measure of non-Gaussianity, is defined as

J(p_{X}_{ⱼ}_{(τ)})=h(σ_{X}_{ⱼ}_{(τ)})−ĥⱼ^{Δ}(τ) (8)

where p_{X}_{ⱼ}_{(τ)} is the amplitude probability distribution defined above, h(σ_{X}_{ⱼ}_{(τ)} is the differential entropy of a Gaussian distribution with the same variance as p_{X}_{ⱼ}_{(τ)} and ĥⱼ^{Δ}(τ) is the bias corrected differential entropy of p_{X}_{ⱼ}(τ) as already defined. Negentropy is calculated in order to determine to what extent variations in hⱼ(τ) are driven by changes in variance or non-Gaussianity.

D. Data Analysis Results

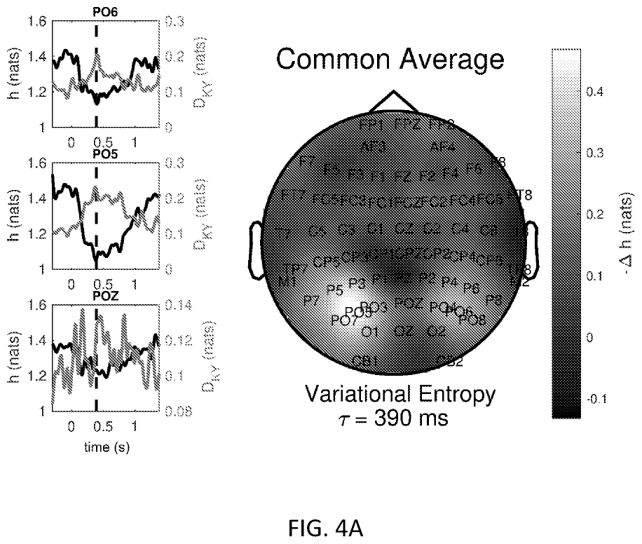

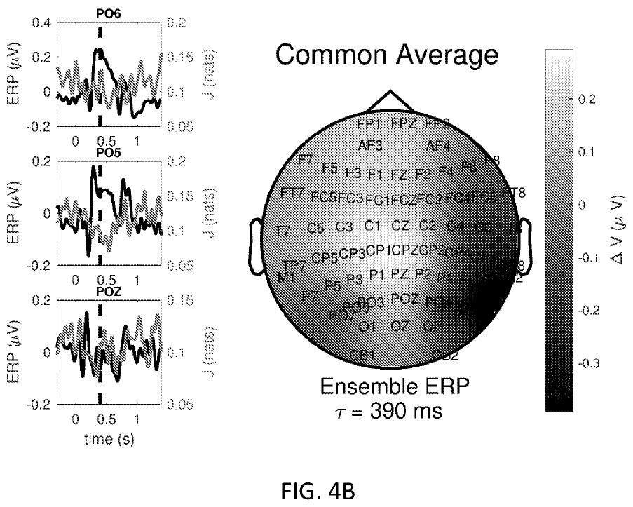

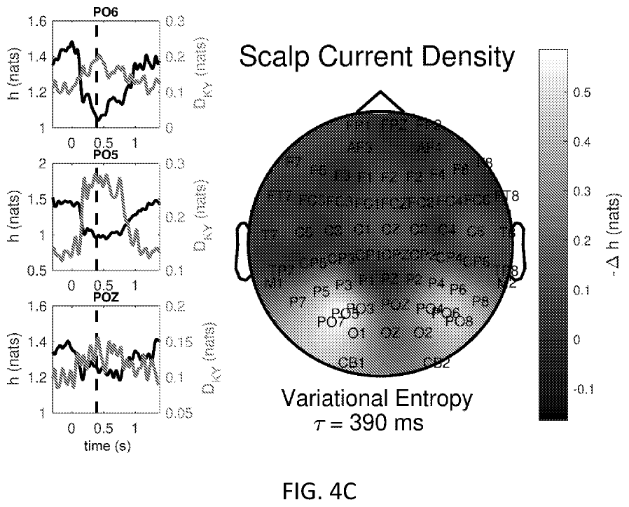

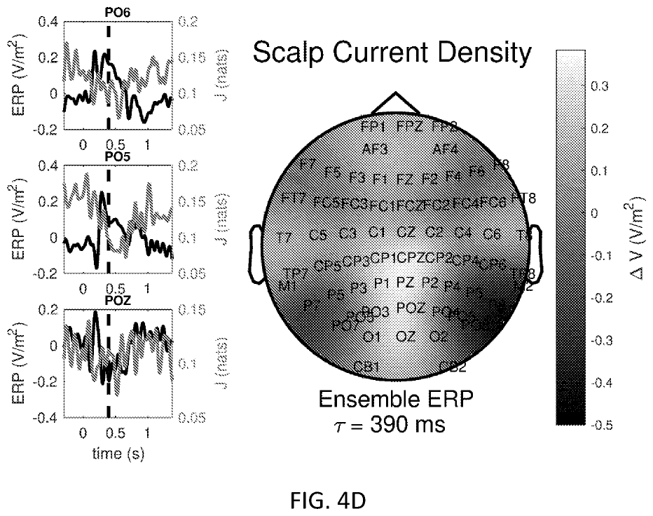

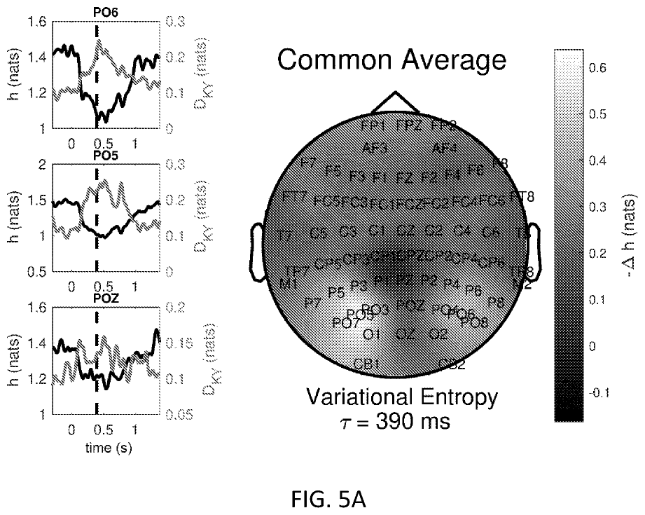

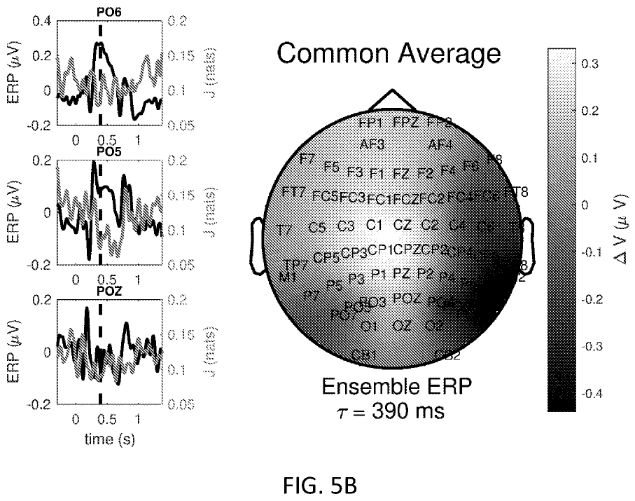

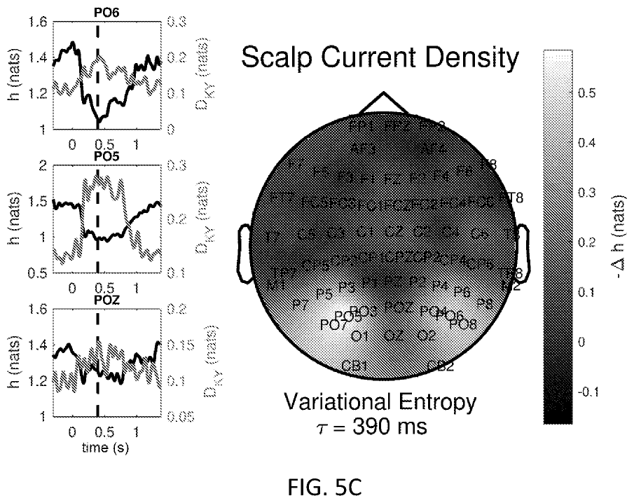

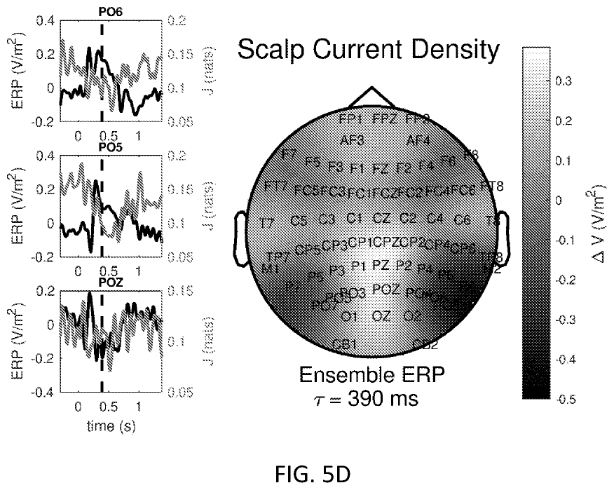

FIG. 4A to FIG. 10B show initial results of evaluating differential entropy and the other measures across trials. FIG. 4A to FIG. 4D and FIG. 5A to FIG. 5D show topographic maps of baseline changes in differential entropy according to EQ.6, −δĥⱼ^{Δ}(τ), for a representative subject. FIG. 4A to FIG. 4D use baseline uncorrected epochs as the basis for all measures. We have shown a single frame of the animated map at a latency of 0.39 s post-stimulus. In contrast FIG. 5A to FIG. 5B use de-meaned epochs. As can be seen the differential entropy in channels PO5, PO6 and POz, for two widely used electrode derivations (Common Average and Laplacian-referenced) rapidly decreases from a baseline following stimulus presentation reaching a minimum at a latency of approximately 0.39 s. This minimum topographically corresponds with a maximal reduction in differential entropy in parietal electrodes—a result apparently consistent with the results of other neuroimaging studies investigating topographic changes in brain activity during reading.

FIG. 4A and FIG. 4C depict coloured topographic plots that show the spatial pattern of variations of differential entropy (in nats) from a pre-stimulus baseline (here referred to a “Variational Entropy”/ΔH) at 390 ms after stimulus presentation (time of minimum post-stimulus differential entropy as indicated by vertical red line in accompanying subplots (i), (ii) and (iii) to left and right). These accompanying subplots show the differential entropy, the Kullback-Leibler divergence (D_{KL}[Xⱼ(τ)>0∥ⱼ(τ≤0)], with respect to a pre-stimulus baseline) for three selected channels/electrodes: PO6, PO5, and POz. The data/measurements in FIG. 4A to FIG. 4D are uncorrected baseline epoch data/measurements.

FIG. 4B and FIG. 4D depict coloured topographic plots which show the corresponding standard time ensemble averaged event related potentials together with corresponding subplots which show the time course of negentropy (J(p_{X}_{ⱼ}_{(τ)}) as defined in EQN. 8) and event related potential amplitude for three selected channels/electrodes: PO6, PO5, and POz).

Similarly, FIG. 5A to FIG. 5D and accompanying subplots present topographic data/measurements and estimates of differential entropy (here labelled H), and other information theoretic quantities as a function of stimulus latency; this time for de-meaned epochs.

The important features to note from a review of FIG. 4A to FIG. 4D, and FIG. 5A to FIG. 5D and accompanying subplots are 1) the transient decrease in differential entropy following stimulus presentation and 2) the spatial heterogeneity of the magnitude changes in differential entropy. These results are based on approximately 300 stimulus presentations.

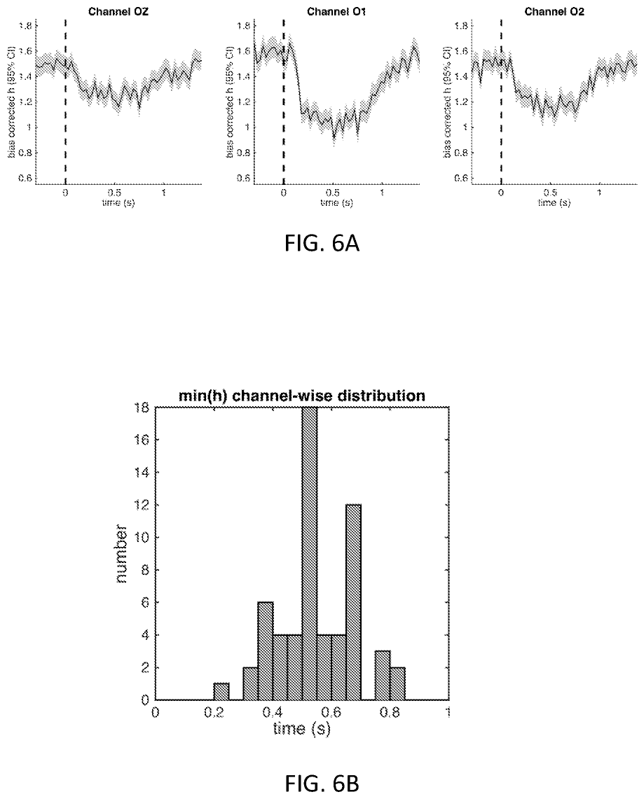

FIG. 6A and FIG. 6B show plots of bias corrected differential entropy for three selected channels for the same subject of FIG. 4A to FIG. 4D and FIG. 5A to FIG. 5D that include the channel (O1) that has the maximal change in entropy following the presentation of the stimulus.

Furthermore, the post-stimulus changes in differential entropy are more clearly seen as mean bias corrected differential entropy represented by a solid line in FIG. 6A (i) Channel OZ (ii) Channel O1 and (iii) Channel O2. The bootstrap 95% confidence intervals for differential entropy are also plotted (shading). These changes in post-stimulus differential entropy (together with 95% confidence intervals) are shown for a silent/passive reading task for a single typical subject for electrode channels Oz, O1 and O2.

The important things to notice in these plots are 1) the transient post-stimulus reduction in differential entropy and 2) the magnitude variations in the temporal changes of differential entropy across electrodes, with considerable variation in the timing of the channel-wise differential entropy minima. FIG. 6B shows the distribution of latency times, across all channels, for which the differential entropy was at its minimum.

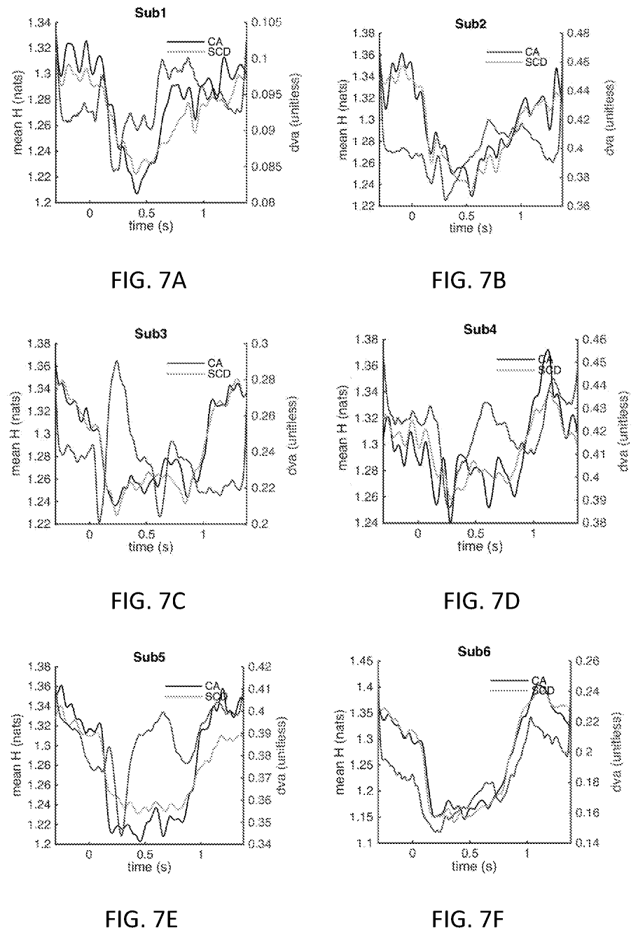

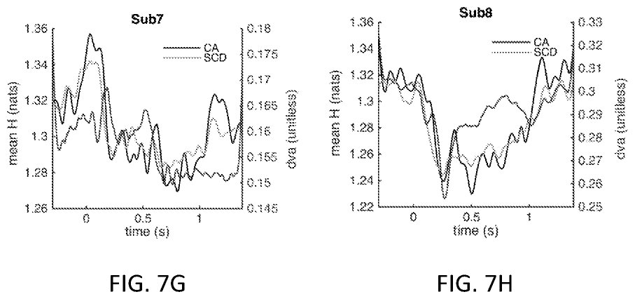

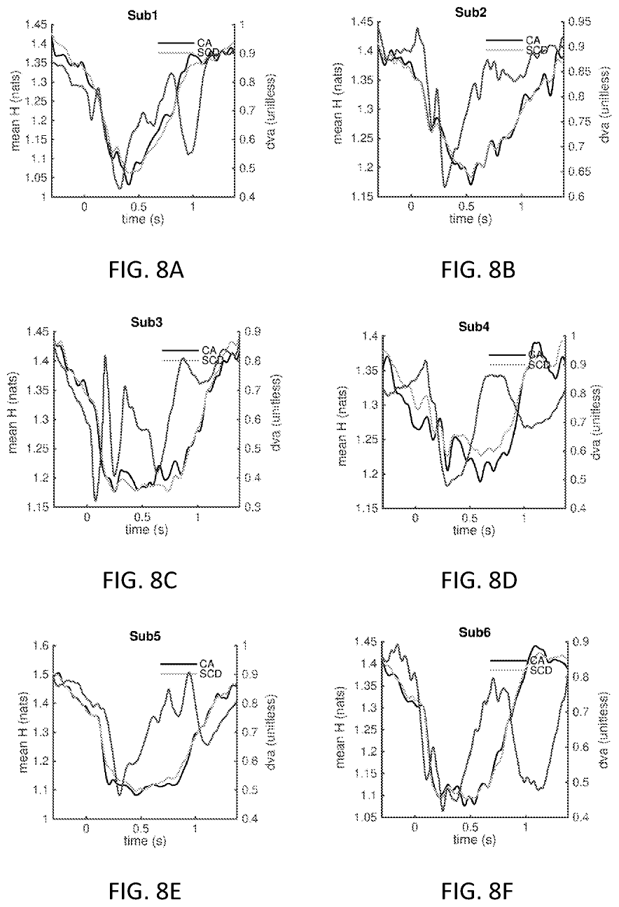

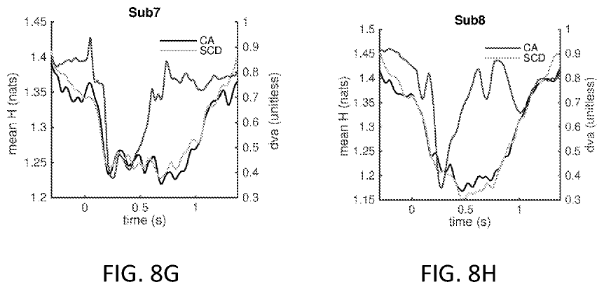

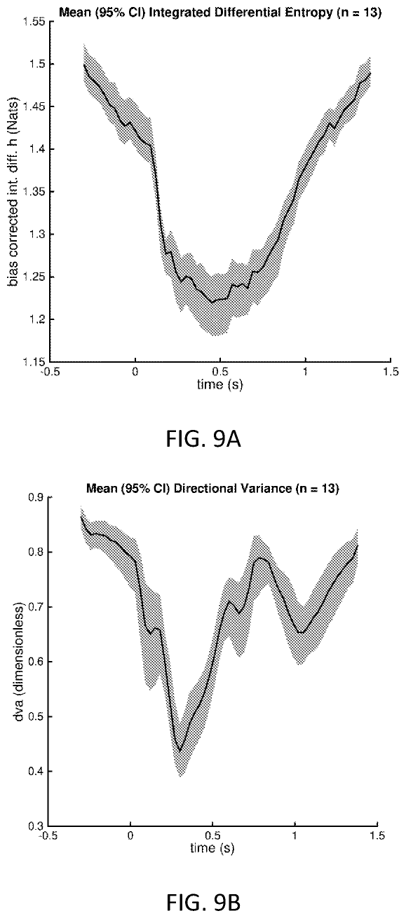

FIG. 7A to FIG. 7H and FIG. 8A to FIG. 8H show both the differential entropy averaged over all electrodes (which we will subsequently refer to as “space integrated” differential entropy) and the dva for all subjects for zero-meaned and baseline uncorrected data. For both baselines a clear reduction in the “space integrated” differential entropy occurs within the first 0.2-0.3 s post stimulus. This response becomes more uniform for zero-meaned data. There is substantial similarity in the time course of this response for both CA referenced and SCD calculated data. FIG. 9A and FIG. 9B illustrate the mean “space integrated” differential entropy and the dva, together with their bootstrap calculated confidence intervals, across all participants.

FIG. 9A depicts the mean integrated differential entropy together with bootstrap calculated confidence intervals (shaded) across all participants.

FIG. 9B depicts the dva, together with bootstrap calculated confidence intervals (shaded), across all participants.

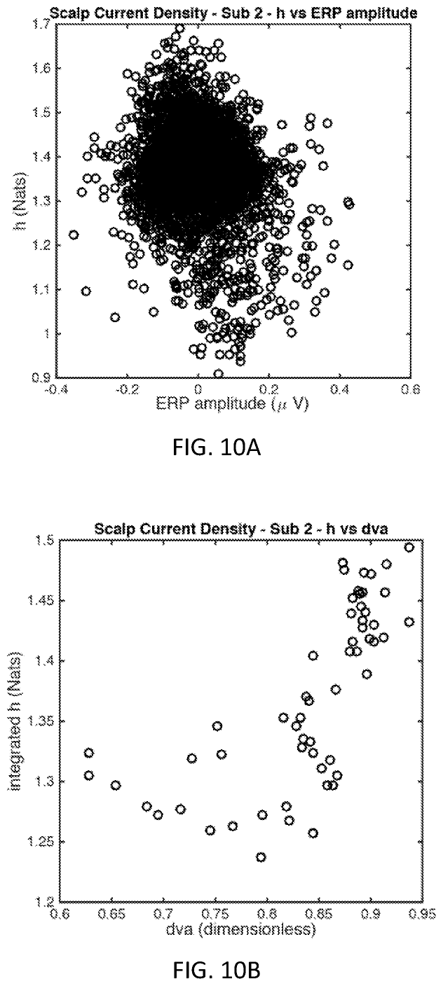

FIG. 10A shows correlations between the bias corrected differential entropy and the amplitude of the ensemble averaged ERPs over all channels and time interval (−0.3, 1.4) s for the subject of the previous figures. It is noted that no clear correlation is seen and is typical of the result seen when examined for other subjects. In other words, estimated changes in differential entropy are not related, and thus independent, of the changes in ensemble averaged ERP amplitude.

FIG. 10B shows the correlation between the integrated (spatially averaged) differential entropy and the dva over the time interval (−0.3, 1.4) s. A weak positive correlation between the integrated h^and dva is seen.

E. Summary of Results and Conclusions Drawn

In summary Example 1 has provided clear illustration that the probability density function p_{X}_{ⱼ}_{(τ)}(xⱼ; τ), as disclosed and defined, changes in response to stimulus presentation as quantified by information theoretic measures that include differential entropy, “space averaged” differential entropy, Kullback-Leibler divergence and negentropy. Further, such quantifiable changes are seen to be independent of corresponding time ensemble derived changes in ERP amplitude.

### EXAMPLE 2

### Differentiating Stimulus Activated Changes:

In a further example, it is demonstrated that informational measures calculated from p_{X}_{ⱼ}_{(τ)}(xⱼ; τ) can effectively quantify trial-by-trial variability and provide a specific interpretation of evoked responses, and importantly can also differentiate the electrophysiological response of different classes of stimuli (lexically vs semantically variable visual word stimuli).

A. Participants and Task

| Participants | 26 (15 males, 9 females, 2 unspecified) native English speakers with | |
|---|---|---|
| | full vision, no current/history neurological and/or psychiatric disorder | |
| | and not currently taking any pharmacological agent or otherwise | |
| | documented to interfere with normal cognition | |
| Task | Presentation of pseudo-random sequence of words (each visible for | |
| | 500 ms with next stimulus appearing 1500 ms later), to be passively | |
| | viewed (i.e. no response required), that vary on two stimulus (word | |
| | type) dimensions | |
| | i) | High and low lexical neighbourhood. Lexical |
| | | neighbourhood refers to words that have a high or low |
| | | number of overlaps with other words based on their |
| | | orthographic and phonological characteristics. For |
| | | example, the word cat has a high lexical neigbourhood |
| | | because there are many other words that are written or |
| | | sound similar such as bat, rat, hat. |
| | ii) | High and low semantic complexity. Semantic complexity |
| | | specifies the number of perceptual features derived from |
| | | their meaning - such as distinctiveness, relatedness and |
| | | concreteness (i.e. elicits a sensory perception). For |
| | | example, a word with high semantic complexity would be |
| | | deodorant as it will evoke a dense population of |
| | | semantically related words such as mouthwash, |
| | | shampoo, lotion, odour, cleanliness. |

B. EEG recording and Preprocessing

| | |
|---|---|
| Equipment | Neuroscan 64 channel SynAmps2/RT EEG recording system |
| Sensor location | Extended 10-20 electrode placement (64 channel waveguard ™ cap) |
| Recording | 1000 Hz sampling rate |
| configuration | common average referencing |
| | all electrode impedance <10 kΩ |
| | bandpass filtering 0.5-35 Hz |
| Data | artefact rejection (visual and independent components |
| preprocessing | analysis) performed using the FieldTrip software toolbox |
| | http://www.fieldtriptoolbox.org/ for MATLAB |
| | no prestimulus baseline correction |
| | data epochs were extracted spanning the time period 0.4 s |
| | before stimulus onset to 1.3 s after stimulus |

C. Calculation of the information theoretic measures differential entropy and negentropy An amplitude distribution for each time point corresponding to stimulus latency τ and j-th electrode/sensor/channel/brain location is formed across trials and is used to calculate the differential entropy, as for Example 1. For a continuous probability density function of amplitudes Xⱼ(τ), p_{X}_{ⱼ}_{(τ)}(xⱼ; τ), the differential entropy, hⱼ(τ), is defined as

hⱼ(τ)=∫p_{X}_{ⱼ}_{(τ)}(xⱼ; τ) log p_{X}_{ⱼ}_{(τ)}(xⱼ; τ) dxⱼ (9)

However, because the number of epochs is finite p_{X}_{ⱼ}_{(τ)}(xⱼ; τ), is estimated from binned amplitude data/measurements. So, histograms of amplitude across all trials for a fixed latency time were constructed (FIG. 3). Histogram bin widths are chosen so that a fixed number of bins will cover the range of single trial amplitudes for a given channel. In this way, the trial-by-trial variability for each word condition was quantified. Now, assuming the amplitude Xⱼ(τ), at time τ is partitioned into bins of width A, differential entropy hⱼ(τ) can be calculated as

$$\begin{matrix}{h}_{j}^{Δ} ( τ ) = {H}_{j}^{Δ} ( τ ) + log Δ \\ = - {\sum }_{i} Δ {p}_{{X}_{j} ( τ )} ( i Δ ; τ ) log Δ {p}_{{X}_{j} ( τ )} ( i Δ ; τ ) + log Δ \\ = - {\sum }_{i} Δ {p}_{{X}_{j} ( τ )} ( i Δ ; τ ) log {p}_{{X}_{j} ( τ )} ( i Δ ; τ )\end{matrix} \qquad ( 10 )$$

Where Hⱼ^{Δ}(τ) is the Shannon Entropy of the discrete probability distribution p_{X}_{ⱼ}_{(τ)}(xⱼ; τ). This simple estimate of differential entropy is known to be biased in that it underestimates true entropy. However, a bootstrap bias correction can be used (as for Example 1-EQN. 5), which also allows the calculation of 95% confidence intervals and bias-corrected negentropy. Negentropy is also calculated to determine to what extent variation in hⱼ(τ) is driven by changes in variance or non-Gaussianity. As for the case of Example 1 negentropy is calculated as

J(pₓ_{ⱼ}_{(τ)})=h(σ_{X}_{ⱼ}_{(τ)})−ĥⱼ^{Δ}(τ) (11)

where p_{X}_{ⱼ}_{(τ)} is the amplitude probability distribution defined above, h(σ_{X}_{ⱼ}_{(τ)}) is the differential entropy of a Gaussian distribution with the same variance as p_{X}_{ⱼ}_{(τ)} and ĥⱼ^{Δ}(τ) is the bias corrected differential entropy of p_{X}_{ⱼ}_{(τ)} as already defined.

The mean event-related potential (ERP) were calculated for all trials included in each condition for each electrode separately, as per the standard ensemble averaging approach as previously described. ERPs were then normalized into Z-scores to enable a consistent treatment with respect to the other calculated measures (of note unnormalized ERP amplitudes were also calculated, which produced essentially identical results in all subsequent statistical analyses as outlined below).

D. Statistical Analysis: Time-Windows and Electrode Clusters

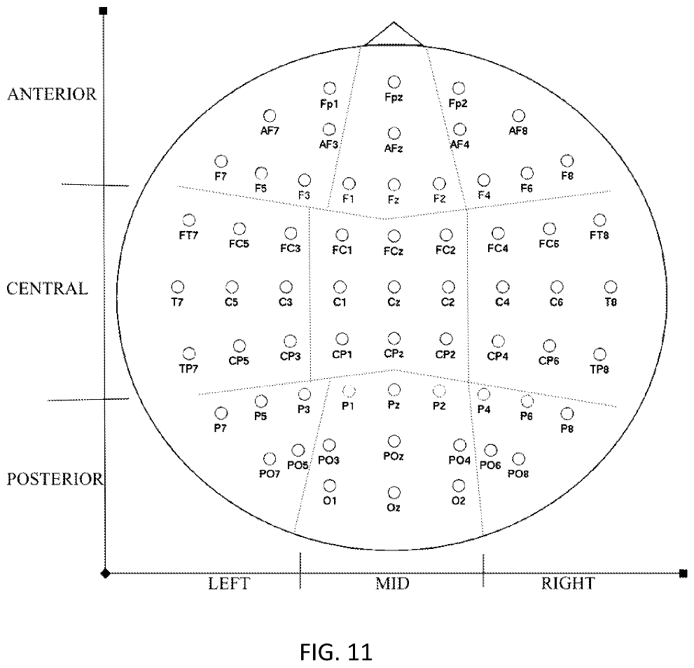

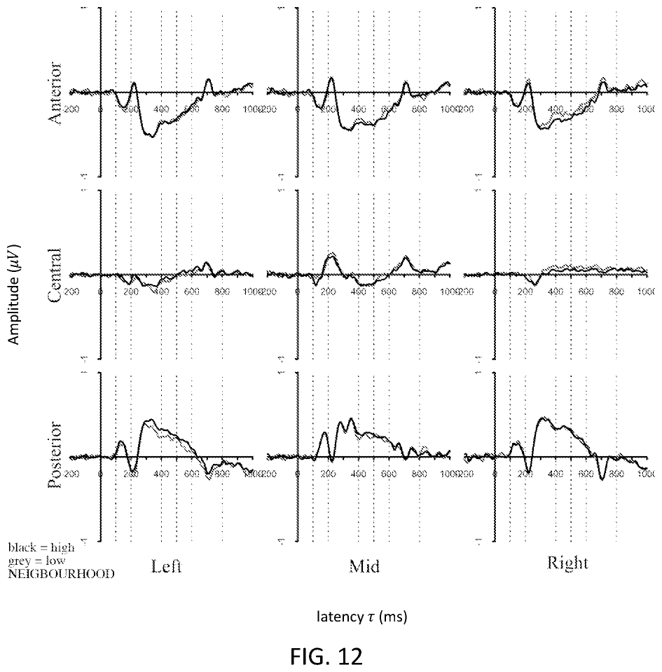

After ERP calculation, data was visually inspected for time-windows of interest to run statistical analyses on, using ERP component literature to guide selection. ERP plots (FIG. 11 and FIG. 12) show clear early lexical components with a negative peak at 100 ms (N100) and a positive peak at roughly 210 ms. A time window was set to capture the full breadth of the N100 with respect to reviewed literature on early lexical access. Inspection also revealed a wide negative trough from roughly 250 ms to 650 ms, where a negative dip of interest occurs at 450 ms. ERP literature suggests a range of word-related semantic and memory processes are associated with a negative peak at 400 ms, so an analysis window was set between 400-500 ms. Finally, a late positive component (LPC) window of 600-800 ms was used to capture any late-processing of word-stimuli. On this basis three time windows were thus defined over which statistical comparisons would be made i) τ=[100, 200] ms ii) τ=[400, 500] ms and iii) τ=[600, 800] ms.

Subsets of electrodes were also defined to simplify statistical analysis. Based on established convention in the analysis of cognitive ERPs, the 64-channel electrode array was divided into clusters of electrodes to investigate regions where high and low exemplars for each manipulation differ significantly in ERP amplitude, differential entropy and negentropy. Three clusters were made along the sagittal axis; anterior, central and posterior. Another three clusters were made across the lateral axis; left, middle and right (FIG. 11).

On the basis of the time windows and electrode clusters so defined analyses were run using R-statistical package using the afex library. ANOVAs used repeated measures with a type III sum of squares, and where the assumption of sphericity was violated, used a Greenhouse-Geisser correction. Data/measurement analyses were run for each time-window of interest using a 3 sagittal (Anterior/Central/Posterior)×3 lateral (Left/Mid/Right)×2 word-type (high/low) ANOVA. For significant main effects, comparisons were run to locate which specific cluster showed a significant word difference.

E. Data Analysis Results

Two participants had to be removed entirely from analysis as more than 50% of their data was unusable. After pre-processing, 79% of trials were included on average per participant (See Table 1. for mean number of trials included for each condition).

TABLE 1
Number of trials for each word condition for Example 2.

| Word Manipulation Condition | Mean Trials Included (SD) |
|---|---|
| High Lexical | 89.00 (6.33) |
| Low Lexical | 89.42 (7.15) |
| High Semantic | 88.58 (6.62) |
| Low Semantic | 90.08 (5.31) |

### Event Related Potential (ERP) Amplitude

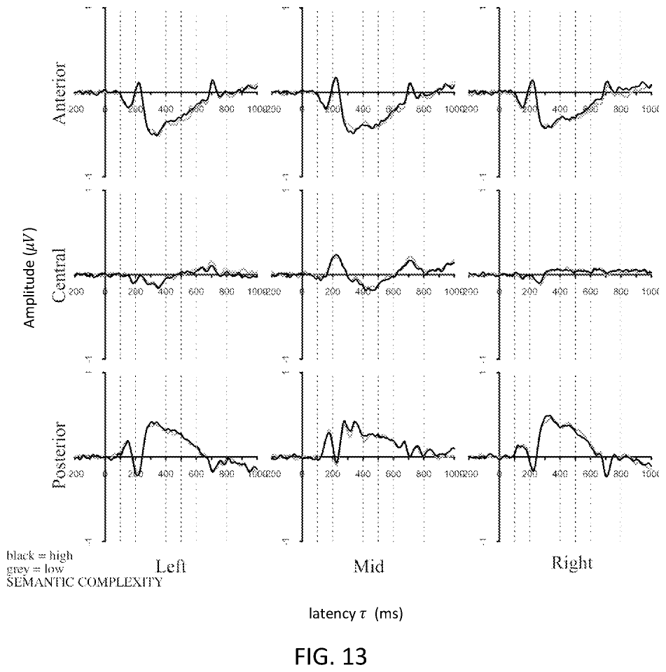

For both the lexical neighbourhood and semantic complexity feature manipulation, clear ERP components of N100, P200, N400 and Late positive components (LPC) are evident in the front electrodes (FIG. 12 and FIG. 13). In the posterior electrodes, these components are still apparent, however due to the common average reference used the voltage of these components is flipped, as the voltage must sum to zero, causing any negativity to be balanced by equivalent positivity.

Lexical Neighbourhood Word Type Condition

| Time window | Remarks |
|---|---|
| τ = [100, 200]ms | No significant word type difference or interaction |
| | was found for this time window. |
| τ = [400, 500]ms | A significant interaction between word-type and lateral electrode |
| | regions was found (F(2, 46) = 3.84, p = 0.003. Comparisons using left, |
| | middle and right electrode clusters showed a significant difference |
| | between high and low lexical neighbourhood words in the right |
| | (t(50.88) = 2.807, p = 0.007), but not in the left |
| | (t(50.88) = −1.75, p = 0.09) or middle |
| | (t(50.88) = −0.37, p = 0.71) electrode clusters. In the right |
| | electrodes, high lexical neighbourhood words showed a larger N400 |
| | deflection than low lexical neighbourhood words (FIG. 12). |
| τ = [600, 800]ms | No significant word type difference or interaction |
| | found for this time window. |

Semantic Complexity Word Type Condition

| Time window | Remarks |
|---|---|
| τ = [100, 200]ms | No significant word type difference or interaction |
| | was found for this time window. |
| τ = [400, 500]ms | No significant word type difference or interaction |
| | was found in this time window. |
| τ = [600, 800]ms | A significant interaction between word-type, laterality and sagittal |
| | position (F(4, 92) = 3.19, p = 0.02) was found. Comparisons |
| | across all electrode positions showed a significant difference |
| | between words from the high and low semantic feature manipulation |
| | in the right anterior electrode cluster (t(91.64) = 2.176, |
| | p = 0.03), where words from the high category show larger |
| | late positive potential amplitudes than those from the low |
| | category in this electrode cluster. |

### Differential Entropy

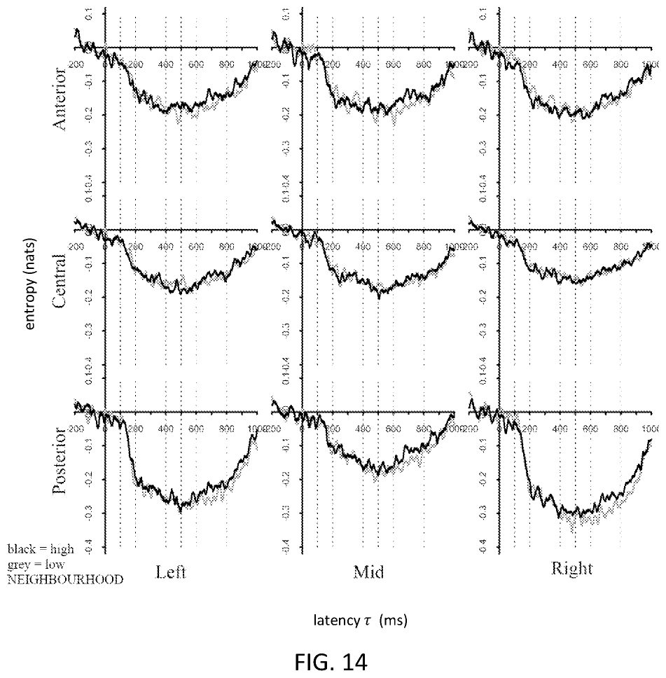

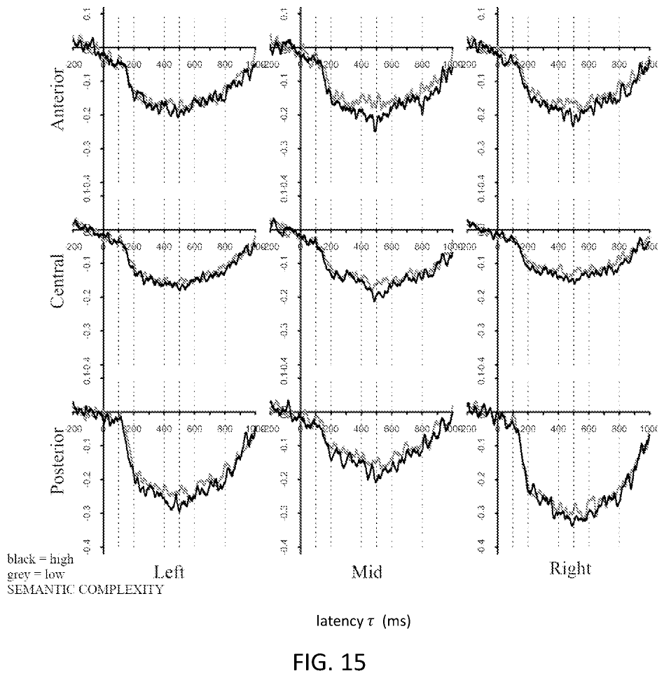

Trial-by-trial variability, calculated as differential entropy using a fixed bin width, was reduced in all electrode clusters after the presentation of either high or low categories in both lexical and semantic word manipulations (FIG. 14 and FIG. 15). Across all trials, the general trend shows a rapid reduction of differential entropy in the τ=[100, 200] ms window reaching the lowest point of reduction between 400-500 ms, from which the differential entropy trends upwards.

Lexical Neighbourhood Word Type Condition

| Time window | Remarks |
|---|---|
| τ = [100, 200]ms | There was no significant word-type difference or interactions |
| | found in this time-window. |
| τ = [400, 500]ms | A significant interaction between word-type and sagittal electrode |
| | position was found in this time-window (F(2, 46) = 4.47, p = 0.02). |
| | Further comparisons between regions along the sagittal axis (anterior, |
| | central, posterior) showed no significant differences. |
| τ = [600, 800]ms | A significant main effect of word-type was found (F(1, 23) = 5.76, p = |
| | 0.02), where words with low lexical neighbourhood showed greater |
| | reductions in entropy than words with high lexical neighbourhood (FIG. |
| | 14). A significant word-type interaction between sagittal electrode |
| | position and word-type was also found (F(1.58, 36.44) = 5.19, p < |
| | 0.001). Comparisons between regions along the sagittal axis (anterior, |
| | central, posterior) showed a significant difference between high and low |
| | neighbourhood words in the anterior electrodes (t(36.81) = 2.14, |
| | p = 0.04), and posterior electrodes (t(36.81) = −3.42 p = |
| | 0.002) but not in the central electrodes (t(36.81) = −0.774, p = 0.44). |

Semantic Complexity Word Type Condition

| Time window | Remarks |
|---|---|
| τ = [100, 200]ms | A significant main effect for word-type showed a difference between |
| | high and low words from the semantic features manipulation (F(1, 23) = |
| | 5.20, p = 0.03). Words from the high category showed lower differential |
| | entropy than words from the low category (FIG. 15). No significant |
| | interactions with word-type were found in this time-window. |
| τ = [400, 500]ms | This window found the strongest main effect for word-type across both |
| | lexical and semantic word manipulations, showing a large significant |
| | difference between high and low words from the semantic manipulation, |
| | (F(1, 23) = 9.94, p = 0.004). Words with a high number of derived |
| | features showed much lower differential entropy than words with less |
| | features. Further comparisons between all electrode clusters showed a |
| | significant difference in most electrode clusters, the strongest effect |
| | being in the mid-anterior (t(76.31) = 4.38, p < 0.001). A significant |
| | interaction between word-type and sagittal electrode position was also |
| | found (F(2, 46) = 5.06, p = 0.01). Comparisons between regions along |
| | the sagittal axis (anterior, central, posterior) showed a significant |
| | difference between high and low semantic category words in the |
| | anterior(t(32.32) = 3.895, p < 0.001, and posterior (t(32.32) = |
| | 3.068, p = 0.004) groups. |
| τ = [600, 800] ms | There were no significant word-type differences or |
| | interactions found in this time window. |

### Negentropy

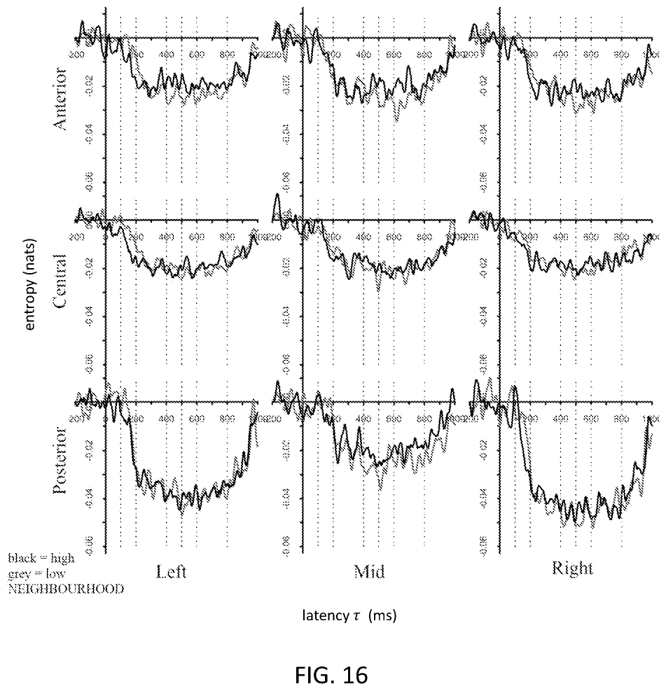

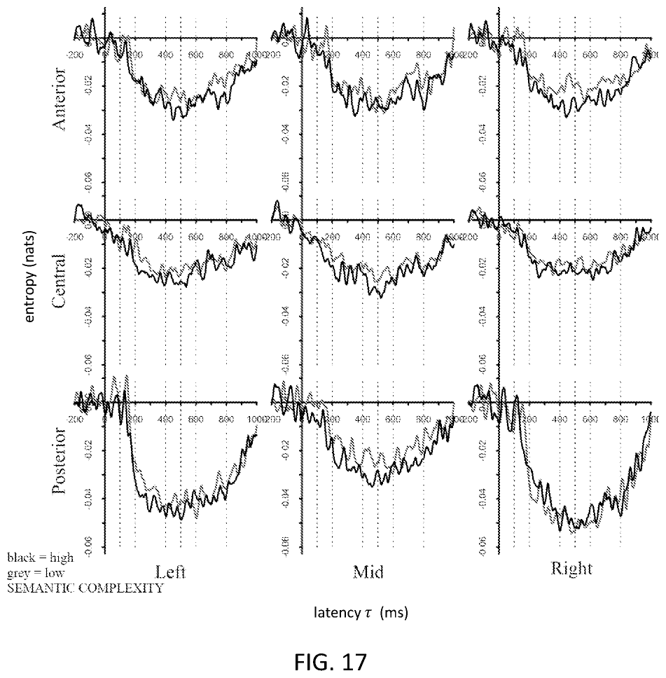

Negentropy for the word manipulations, as a calculation of non-Gaussianity, is plotted in FIG. 15 and FIG. 16. Both graphs show a decline in negentropy as a function of latency from the presentation of word-stimuli. This reaches its lowest point in roughly in the 400-500 ms time window matching that of the differential entropy.

Lexical Neighbourhood Word Type Condition

| Time window | Remarks |
|---|---|
| τ = [100, 200]ms | A significant main effect for word type showed a difference |
| | between high and low lexical neighbourhood words (F(1, 23) = |
| | 4.47, p = 0.05) where words with high lexical neighbourhood |
| | showed lower negentropy scores than words with low |
| | neighbourhood; |
| | A significant interaction between word-type and the lateral |
| | position of electrode clusters was also found. Comparisons |
| | between electrodes in the left, middle and right regions showed a |
| | significant difference between high and low lexical neighbourhood |
| | words in the left (t(37.73) = 2.75, p = 0.009) and |
| | right (t(37.73) = 2.176, p = 0.04) but not in the middle |
| | electrodes (t(37.73) = 0.62, p = 0.54). |
| τ = [400, 500]ms | Did not find any significant word-type differences or interactions. |
| τ = [600, 800]ms | Did not find any significant word-type differences or interactions. |

No significant differences or interactions between words from the semantic complexity feature manipulation were found in any of the predefined time windows (100-200 ms, 400-500 ms, 600-800 ms).

F. Summary of Results and Conclusions Drawn

The only significant ERP lexical neighbourhood word-type difference was in the 400-500 ms window across right-laterality electrodes. Where, words with high lexical neighbourhood caused greater N400 deflections than words with lower lexical neighbourhood. It will be appreciated, by those familiar in the art of cognitive ERP processing and interpretation, that such an N400 effect would generally understood as arising from the high-lexical neighbourhood word type condition facilitating semantic processing.

Similarly, the only significant ERP semantic complexity word type effect differences were found in the 600-800 ms window. Based on the conclusion that positive components occurring over 400-800 ms are associated with the recollection of specific information, which may link higher semantic features to access greater detail in meaning, it can be concluded that words with more semantic features would be expected to show greater amplitudes in positive components as seen here. We have not considered how later positive components may be influenced by semantic richness in visual word recognition tasks.

In contrast the word stimulus effects on changes in differential entropy were much more marked.

All subjects demonstrated a strong reduction in differential entropy, when evaluated across all word type conditions, with reduction onset occurring immediately after stimuli presentation. A strong decrease at 100-150 ms, particularly in posterior electrodes, reaching a minimum at 400-500 ms was found for both lexical and semantic manipulations.

Significant lexical neighbourhood word type differences were reflected in differences in the calculated differential entropy provide support for the contention that changes in differential entropy correspond to meaningful changes in brain activity and reflect important aspects of neural information processing. Lower values of differential entropy were found following the presentation of words with low lexical neighbourhood in the 400-500 ms and 600-800 ms time window, suggesting that orthographically unique words, as expected, are more meaningful. The only significant interaction between word-type and electrode location showed significant word-type differences across the anterior and posterior electrodes in the 600-800 ms window.

Similarly, and again as anticipated, a significant difference between words with high and low semantic complexity was observed in the changes in differential entropy following word presentation. Specifically, words with a high number of semantic features resulted in lower values of differential entropy than words with low semantic features in both the 100-200 ms and 400-500 ms time windows. This supports the central idea that a stimulus, in this case a word, with greater meaning would cause a greater reduction in the uncertainty of recorded electrophysiological activity; and is consistent with the understanding of richness effects in the contemporary literature, where a greater number of semantic features leads to richer meaning of the target word which translates to better semantic processing.

When all the basic perceptual components of the words were held constant a significant difference in the differential entropy between words of high and low semantic richness is noted. An expected significant semantic complexity word type difference occurred in the 400-500 ms time window. The 400-500 ms window clearly illustrates (FIG. 14) that words with high semantic richness were associated with greater reductions of differential entropy. The topography of the identified changes in differential entropy following word presentation are in general agreement with a range of other neuroimaging studies, where a significant semantic complexity word type effect was found across posterior and anterior electrodes.

Finally, as shown in FIG. 15 and FIG. 16 global reductions in negentropy were seen in both lexical neighbourhood and semantic complexity word type stimuli thus demonstrating a decrease in “free” energy indicative of increased dynamical stability, consistent with the settling of a recurrent neural network into a decision state. Such a reduction in negentropy provides support for the notion that stimulus-evoked variation in calculated differential entropy, hⱼ(τ), is most likely driven by non-Gaussianity as opposed to alternate mechanisms that influence trial-by-trial variability such as decreased EEG power or increased phase coherence across trials.

In the Examples described above, the use of an informational measure, such as differential entropy to quantify trial-by-trial neural variability of a stimulus driven evoked potential provides a greater characterization of the neural response. On the basis of the results of Examples 1 & 2, such increases/decreases in differential entropy can reasonably be interpreted as corresponding to increases/decreases in the cognitive “meaning” of the stimuli. This was clearly observed in the findings of early semantic processing of word-type stimuli, where words of high complexity were associated with greater reductions in differential entropy than words of lower semantic complexity. In the context of typical cognitive paradigms such as the Oddball sequence, differential entropy can be profitably used characterize trial-by-trial variability in a range of neurological and psychiatric disorders that include ADHD, dyslexia and psychosis.

The spatio-temporal patterns of changes in differential entropy, and any other information theoretic quantities derived from p_{X}_{ⱼ}_{(τ)}(Xⱼ; τ), with respect to given stimuli or classes of stimuli, are predicted to be individually specific and thus expected to represent some form of “cognitive fingerprint”. On this basis longitudinally assessed deviations from such individually “normative” patterns will be utilized to objectively diagnose pathological functional neurologic and psychiatric states.

Other information theoretic measures, calculated from p_{X}_{ⱼ}_{(τ)}(xⱼ; τ), can also be employed for such diagnostic purposes. These measures will include, but are not restricted to, pairwise symmetric mutual information I[Xⱼ(τ); X_{j′}(τ′)], negentropy, asymmetric transfer entropy, conditional entropy, relative entropy/Kullback-Leibler divergence, joint entropy and multiscale entropy. Such information theoretic measures are well known to be able to specify important aspects of p_{X}_{ⱼ}_{(τ)}(xⱼ; τ) in particular its systematic interdependencies over time and space. Such measures will be central to systematically determining task and individual differences, outcomes that are signally important from a diagnostic perspective.

It can be appreciated that when considering brain activity monitoring, p_{X}_{ⱼ}_{(τ)}(xⱼ; τ) may be configured to be serially estimated in time from a plurality of sessions for a specified subject. Each session may comprise measurements of brain activity for a plurality of repeated stimuli. Sessions may be spaced apart at intervals selected from a plurality of hours, plurality of days, plurality of months or a plurality of years. Similarity, hⱼ(τ) can be longitudinally estimated from a plurality of sessions for a specified subject wherein each session comprises measurements of brain activity for a plurality of repeated stimuli and the sessions are spaced apart at intervals selected from a plurality of hours, plurality of days, plurality of months or a plurality of years.

The empirical estimation of these quantities can be achieved by multiple existing methods (e.g. integrated kernel density estimates, Gaussian copula) etc.

Advantageously the present disclosure can define an “information fingerprint” for each stimulus/class of stimulus, for each individual at a moment in time and for a given spatio-temporal scale (depending on the brain-related signal modality being used to observe brain activity).

Any change in this “information fingerprint” in the context of learning and normal ongoing activity, will provide important information for the ongoing assessment of neurologic and psychiatric function in health and disease. Accordingly, the method and systems of the present disclosure can provide a meaningful objective instrument for the longitudinal assessment of brain information processing. Furthermore, it may be applied to an assessment of an individual subject's brain function and as such can be used for a wide range of neurodiagnostic monitoring purposes, including monitoring disease progression, monitoring levels of sedation (e.g. under anaesthesia, to prevent intra-operative recall or awakening), monitoring alertness/arousal/attention as well as furnishing a method for establishing a brain-computer interface (BCI).

It would be appreciated that by contrast, the current state-of-the-art techniques (principally reliant on ensemble averaged event related potentials, event related de-/synchronisation) provide measures that are too poorly differentiated within and across individuals to provide a meaningful objective longitudinal assessment of brain information processing. Compared to the state-of-the-art extraction of stimulus evoked (event related potentials) and induced (event related de-/synchronisation) electroencephalographic activity the present disclosure is not restricted to the calculation of first and second moments of frequency band limited brain activity.

It would be appreciated that the above embodiments are described by way of example only. Many variations are possible without departing from the scope of the invention as defined in the appended claims. For clarity of explanation, in some instances the present technology may be presented as including individual functional blocks including functional blocks comprising devices, device components, steps or routines in a method embodied in software, or combinations of hardware and software.

Methods according to the above-described examples can be implemented using computer-executable instructions that are stored or otherwise available from computer readable media. Such instructions can comprise, for example, instructions and data which cause or otherwise configure a general-purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, Universal Serial Bus (USB) devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

1. A method for constructing a representation of changes in the state of responsiveness of a mammalian subject's brain to a plurality of repeated sensory stimuli, including:
acquiring a plurality of brain activity measurements of a subject over a plurality of pre-determined time periods, wherein each of said measurements are of the subject's brain activity preceding and following the presentation of a sensory stimulus;
using a processor to evaluate variability in the acquired brain activity measurements to a plurality of repeated sensory stimuli; and
generating a report of the changes in brain responsiveness states from the variability in said brain activity measurements over the plurality of predetermined time periods.

2. The method of claim 1 wherein the variability in said brain activity is evaluated according to a probability density function
pₓ_{ⱼ}_{(τ)}(xⱼ; τ)
for brain activity measurements corresponding to the random variable Xⱼ(τ), recorded at a plurality of physical brain locations, each indexed by the integer j, at a time τ, with respect to the presentation of stimuli at time τ=0.

3. (canceled)

4. The method of claim 2 wherein p_{X}_{ⱼ}_{(τ)}(xⱼ; τ) is serially estimated in time from a plurality of sessions for a specified subject, wherein each session comprises measurements of brain activity for a plurality of repeated stimuli and the sessions are spaced apart at intervals selected from a plurality of hours, plurality of days, plurality of months or a plurality of years.

5. The method of claim 4 wherein the serial estimation of p_{X}_{ⱼ}_{(τ)}(xⱼ; τ) for a specified subject provides an indication of changes in brain function between sessions.

6. The method of claim 2 wherein the brain activity is time-ensemble estimated stimulus evoked activity, ERPⱼ(τ), which can be variously estimated as one of the following functions:
(i) ERPⱼ(τ)=E [Xⱼ(τ)], where E[·] is the expectation operator; or
(ii) ERPⱼ(τ)=median[Xⱼ(τ)]; or
(iii) ERPⱼ(τ)=mode[Xⱼ(τ)]

7. The method of claim 2 wherein p_{X}_{ⱼ}_{(τ)}(xⱼ; τ) is empirically estimated on frequency band limited brain activity measurements.

8. The method of claim 2 wherein the serial assessment of p_{X}_{ⱼ}_{(τ)}(xⱼ; τ) for a specified subject provides an indication of brain function over the cumulative sessions.

9. The method of claim 1 wherein the brain activity measurements are acquired by a modality selected from the group of modalities comprising electrocorticogram (ECoG), electroencephalogram (EEG), magnetoencephalogram (MEG), blood oxygen level dependent (BOLD) functional magnetic resonance (fMRI) and near infrared spectroscopy (NIRS).

10. (canceled)

11. The method of claim 1 wherein the measurements are acquired from a plurality of brain locations for a plurality of predetermined time periods.

12. The method of claim 1 wherein one brain responsiveness state is differential entropy determined according to the equation:
hⱼ(τ)=∫p_{X}_{ⱼ}_{(τ)}(xⱼ; τ) log p_{X}_{ⱼ}_{(τ)}(xⱼ; τ) dxⱼ
where hⱼ(τ) is the differential entropy at a time T after the presentation of a stimulus, for a physical brain location specified by the index j.

13. The method of claim 12 further including:
empirically estimating the differential entropy from a finite number of samples; and
defining changes in differential entropy with respect to a baseline reference value.

14. (canceled)

15. (canceled)

16. The method of claim 10 wherein hⱼ(τ) is longitudinally estimated from a plurality of sessions for a specified subject wherein each session comprises measurements of brain activity for a plurality of repeated stimuli and the sessions are spaced apart at intervals selected from a plurality of hours, plurality of days, plurality of months or a plurality of years.

17. The method of claim 16 wherein the longitudinal estimates of hⱼ(τ) indexed by j are plotted topographically with respect to physical brain location.

18. The method of claim 2 wherein the variability of said brain activity is used to derive one or more quantitative information theoretic measures representative of brain responsiveness state.

19. The method of claim 18 wherein the one or more quantitative information theoretic measures representative of brain function are selected from the group comprising negentropy, differential entropy, “space averaged” differential entropy, Kullback-Leibler divergence and negentropy transfer entropy, mutual information, relative entropy and multiscale entropy.

20. (canceled)

21. The method of claim 18 wherein the one or more quantitative information theoretic measures representative of brain function are longitudinally estimated from a plurality of sessions for a specified subject, wherein each session comprises measurements of brain activity for a plurality of repeated stimuli and the sessions are spaced apart at intervals selected from a plurality of hours, a plurality of days, a plurality of months or a plurality of years.

22. (canceled)

23. (canceled)

24. A system for representing the changes in responsiveness states of a mammalian subject's brain in response to a plurality of repeated sensory stimuli, comprising:
an acquiring module including a processor configured for acquiring a plurality of brain activity measurements of a subject over a plurality of pre-determined time periods, wherein each of said measurements are of the subject's brain activity preceding and following the presentation of a sensory stimulus;
an evaluating module including a processor configured for receiving said brain activity measurements and evaluating variability thereof over the plurality of the predetermined time periods; and
a determining module including a processor configured for determining changes in brain responsiveness states from the variability over the plurality of predetermined time periods and generating a report therefrom.

25. The system of claim 24 wherein the variability is evaluated by a processor in the evaluating module configured to utilise a probability density function
p_{X}_{ⱼ}_{(τ)}(xⱼ; τ)
for brain activity corresponding to the random variable Xⱼ(τ), recorded at a plurality of physical brain locations, each indexed by the integer j, at a time τ, with respect to the presentation of stimuli at time τ=0.

26. (canceled)

27. The system of claim 25 wherein p_{X}_{ⱼ}_{(τ)}(xⱼ; τ) is serially estimated in time from a plurality of sessions for a specified subject, wherein each session comprises measurements of brain activity for a plurality of repeated stimuli and the sessions are spaced apart at intervals selected from a plurality of hours, plurality of days, plurality of months or a plurality of years.

28. (canceled)

29. The system of claim 24 wherein one brain responsiveness state is differential entropy calculated by the processor of the determining module according to the equation:
hⱼ(τ)=∫p_{X}_{ⱼ}_{(τ)}(xⱼ; τ)log p_{X}_{ⱼ}_{(τ)}(xⱼ; τ) dxⱼ
where hⱼ(τ) is the differential entropy at a time T after the presentation of a stimulus, for a physical brain location specified by the index j.

30. A non-transitory computer readable medium comprising program instructions that, when executed by one or more processors, implement a method comprising:
acquiring a plurality of brain activity measurements of a subject over a plurality of pre-determined time periods, wherein each of said measurements are of the subject's brain activity preceding and following the presentation of a sensory stimulus;
using a processor to evaluate variability in the acquired brain activity measurements to a plurality of repeated sensory stimuli; and
generating a report of the changes in brain responsiveness states from the variability in said brain activity measurements over the plurality of predetermined time periods.

31. The method of claim 11 wherein hⱼ(τ) is longitudinally estimated from a plurality of sessions for a specified subject wherein each session comprises measurements of brain activity for a plurality of repeated stimuli and the sessions are spaced apart at intervals selected from a plurality of hours, plurality of days, plurality of months or a plurality of years.
